SEQUENCE LISTING (18 sequences)

SEQ ID NO: 1 - protein, 529 aa, Tyrosinase Precursor [sequence omitted]

SEQ ID NO: 2 - protein, 118 aa, MART-1
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 3 - protein, 223 aa, Synovial Sarcoma
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGNREAQEKEERRGTAHRWSSQNTHNIGRFSLSTSMGAVHGTPKTITHNRDPKGGNMPGPTDCVRENSW

SEQ ID NO: 4 - protein, 750 aa, Folate Hydrolase
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - protein, 309 aa, MAGE-1
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 6 - protein, 314 aa, MAGE-3
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 7 - protein, 180 aa, Autoimmunogenic Cancer
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 8 - protein, 509 aa, Preferentially expressed antigen in melanoma
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 9 - protein, 1255 aa, Receptor protein-tyrosine kinase [sequence omitted]

SEQ ID NO: 10 - DNA, 2384 bp, Homo sapiens
tattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaacataggcctatcccactggtgggatacgagccaattcgaaagaaaagtcagtcatgtgcttttcagaggatgaaagcttaagataaagactaaaagtgtttgatgctggaggtgggagtggtattatataggtctcagccaagacatgtgataatcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttatttt

SEQ ID NO: 11 - DNA, 1524 bp, Human melanoma antigen recognized by T-cells (MARA
agcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctg

SEQ ID NO: 12 - DNA, 1466 bp, Homo sapiens synovial sarcoma, X breakpoint 2 (SSA
gcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggaaatagggaggcccaagaaaaggaagagagacgcggaacagctcatcggtggagcagtcagaacacacacaacattggtcgattcagtttgtcaacttctatgggtgcagttcatggtacccccaaaacaattacacacaacagggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaa

SEQ ID NO: 13 - DNA, 2653 bp, Homo sapiens folate hydrolase (prostate-specific A
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 14 - DNA, 2420 bp, Human antigen (MAGE-1) gene, complete cds
ggatccaggccctgccaggaaaaatataagggccctgcgtgagaacagagggggtcatccactgcatgagagtggggatgtcacagagtccagcccaccctcctggtagcactgagaagccagggctgtgcttgcggtctgcaccctgagggcccgtggattcctcttcctggagctccaggaaccaggcagtgaggccttggtctgagacagtatcctcaggtcacagagcagaggatgcacagggtgtgccagcagtgaatgtttgccctgaatgcacaccaagggccccacctgccacaggacacataggactccacagagtctggcctcacctccctactgtcagtcctgtagaatcgacctctgctggccggctgtaccctgagtaccctctcacttcctccttcaggttttcaggggacaggccaacccagaggacaggattccctggaggccacagaggagcaccaaggagaagatctgtaagtaggcctttgttagagtctccaaggttcagttctcagctgaggcctctcacacactccctctctccccaggcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttcactggctcttttcttctccatgcactgagcatctgctttttggaaggccctgggttagtagtggagatgctaaggtaagccagactcatacccacccatagggtcgtagagtctaggagctgcagtcacgtaatcgaggtggcaagatgtcctctaaagatgtagggaaaagtgagagaggggtgagggtgtggggctccgggtgagagtggtggagtgtcaatgccctgagctggggcattttgggctttgggaaactgcagttccttctgggggagctgattgtaatgatcttgggtggatcc

SEQ ID NO: 15 - DNA, 4204 bp, Human MAGE-3 antigen (MAGE-3) gene, complete cds
acgcaggcagtgatgtcacccagaccacaccccttcccccaatgccacttcagggggtactcagagtcagagacttggtctgaggggagcagaagcaatctgcagaggatggcggtccaggctcagccaggcatcaacttcaggaccctgagggatgaccgaaggccccgcccacccacccccaactcccccgaccccaccaggatctacagcctcaggacccccgtcccaatccttaccccttgccccatcaccatcttcatgcttacctccacccccatccgatccccatccaggcagaatccagttccacccctgcccggaacccagggtagtaccgttgccaggatgtgacgccactgacttgcgcattggaggtcagaagaccgcgagattctcgccctgagcaacgagcgacggcctgacgtcggcggagggaagccggcccaggctcggtgaggaggcaaggtaagacgctgagggaggactgaggcgggcctcacctcagacagagggcctcaaataatccagtgctgcctctgctgccgggcctgggccaccccgcaggggaagacttccaggctgggtcgccactacctcaccccgccgacccccgccgctttagccacggggaactctggggacagagcttaatgtggccagggcagggctggttagaagaggtcagggcccacgctgtggcaggaatcaaggtcaggaccccgagagggaactgagggcagcctaaccaccaccctcaccaccattcccgtcccccaacacccaaccccacccccatcccccattcccatccccacccccacccctatcctggcagaatccgggctttgcccctggtatcaagtcacggaagctccgggaatggcggccaggcacgtgagtcctgaggttcacatctacggctaagggagggaaggggttcggtatcgcgagtatggccgttgggaggcagcgaaagggcccaggcctcctggaagacagtggagtcctgaggggacccagcatgccaggacagggggcccactgtacccctgtctcaaaccgaggcaccttttcattcggctacgggaatcctagggatgcagacccacttcagcagggggttggggcccagccctgcgaggagtcatggggaggaagaagagggaggactgaggggaccttggagtccagatcagtggcaaccttgggctgggggatgctgggcacagtggccaaatgtgctctgtgctcattgcgccttcagggtgaccagagagttgagggctgtggtctgaagagtgggacttcaggtcagcagagggaggaatcccaggatctgcagggcccaaggtgtacccccaaggggcccctatgtggtggacagatgcagtggtcctaggatctgccaagcatccaggtgaagagactgagggaggattgagggtacccctgggacagaatgcggactgggggccccataaaaatctgccctgctcctgctgttacctcagagagcctgggcagggctgtcagctgaggtccctccattatcctaggatcactgatgtcagggaaggggaagccttggtctgagggggctgcactcagggcagtagagggaggctctcagaccctactaggagtggaggtgaggaccaagcagtctcctcacccagggtacatggacttcaataaatttggacatctctcgttgtcctttccgggaggacctgggaatgtatggccagatgtgggtcccctcatgtttttctgtaccatatcaggtatgtgagttcttgacatgagagattctcaggccagcagaagggagggattaggccctataaggagaaaggtgagggccctgagtgagcacagaggggatcctccaccccagtagagtggggacctcacagagtctggccaaccctcctgacagttctgggaatccgtggctgcgtttgctgtctgcacattgggggcccgtggattcctctcccaggaatcaggagctccaggaacaaggcagtgaggacttggtctgaggcagtgtcctcaggtcacagagtagagggggctcagatagtgccaacggtgaaggtttgccttggattcaaaccaagggccccacctgccccagaacacatggactccagagcgcctggcctcaccctcaatactttcagtcctgcagcctcagcatgcgctggccggatgtaccctgaggtgccctctcacttcctccttcaggttctgaggggacaggctgacctggaggaccagaggcccccggaggagcactgaaggagaagatctgtaagtaagcctttgttagagcctccaaggttccattcagtactcagctgaggtctctcacatgctccctctctccccaggccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcactggctcgtttcttttccgttcactcagcatctgctctgtgggaggccctgggttagtagtggggatgctaaggtaagccagactcacgcctacccatagggctgtagagcctaggacctgcagtcatataattaaggtggtgagaagtcctgtaagatgtagaggaaatgtaagagaggggtgagggtgtggcgctccgggtgagagtagtggagtgtcagtgc

SEQ ID NO: 16 - DNA, 752 bp, Human autoimmunogenic cancer/testis antigen NY-ESs
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 17 - DNA, 2148 bp, Homo sapiens preferentially expressed antigen in A
gcttcagggtacagctcccccgcagccagaagccgggcctgcagcgcctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcaaaaaaaaaaaaaaaaaa

SEQ ID NO: 18 - DNA, 4530 bp, Human tyrosine kinase-type receptor (HER2) mRNA, s
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

(19) United States
(12) Patent Application Publication
(10) Pub. No.: US 2009/0148478 A1
(43) Pub. Date: Jun. 11, 2009

(54) COMBINATIONS OF TUMOR-ASSOCIATED ANTIGENS IN COMPOSITIONS FOR VARIOUS TYPES OF CANCERS

(75) Inventors: Chih-Sheng CHIANG, Chatsworth, CA (US); Adrian Ion BOT, Valencia, CA (US); John J.L. SIMARD, West Vancouver (CA); David C. DIAMOND, West Hills, CA (US)

Correspondence Address: Davis Wright Tremaine LLP/Mankind Corporation, 505 MONTOGOMERY STREET, STE. 800, SAN FRANCISCO, CA, 94111-6533, US

(73) Assignee: MANNKIND CORPORATION, Valencia, CA (US)

(21) Appl. No.: 12/253,213

(22) Filed: Oct. 16, 2008

Related U.S. Application Data
(63) Continuation of application No. 10/871,708, filed on Jun. 17, 2004, now abandoned.
(60) Provisional application No. 60/479,554, filed on Jun. 17, 2003.

(51) Int. Cl. A61K 39/00; A61K 31/7088

(52) U.S. Cl. 424/277.1; 514/44

(57) ABSTRACT

Disclosed herein are methods and compositions for inducing an immune response against various combinations of tumor-associated antigens, which can promote effective immunologic intervention in pathogenic processes. Embodiments of the invention disclosed herein are directed to the use of effective combinations of TuAAs for the immunotherapy of patients with various types of cancer. Both immunogenic compositions for inducing an immune response to these combinations of antigens and methods for their use are disclosed.

### CROSS REFERENCE TO RELATED APPLICATIONS

This application claims priority under 35 U.S.C. § 119(e) to U.S. Provisional Application No. 60/479,554, filed on Jun. 17, 2003, entitled COMBINATIONS OF TUMOR-ASSOCIATED ANTIGENS IN COMPOSITIONS FOR VARIOUS TYPES OF CANCERS; the disclosure of which is incorporated herein by reference in its entirety.

### BACKGROUND OF THE INVENTION

1. Field of the Invention

Disclosed herein are methods and compositions for inducing an immune response against various combinations of tumor-associated antigens, which can promote effective immunologic intervention in pathogenic processes.

2. Description of the Related Art

The American Cancer Society has estimated that over one million people get cancer each year, and that approximately one out of every two American men and one out of every three American women will have some type of cancer at some point during their lifetime.

Cancer generally develops when cells in a part of the body begin to grow out of control. Although there are many kinds of cancer, they usually start because of out-of-control growth of abnormal cells.

Normal body cells grow, divide, and die in an orderly fashion. Cancer cells are different in that they continue to grow and divide. Instead of dying, they outlive normal cells and continue to form new abnormal cells.

Usual treatment options for cancer include surgery, radiation therapy, and chemotherapy. A fourth branch of treatment is developing, which is referred to as immunotherapy. Immunotherapies attempt to help the immune system recognize cancer cells, and/or to strengthen a response against cancer cells in order to destroy the cancer. Immunotherapies include active and passive immunotherapies. Active immunotherapies attempt to stimulate the body's own immune system to fight the disease. Passive immunotherapies generally do not rely on the body to attack the disease; instead, they use immune system components (such as antibodies) created outside of the body.

Despite the various types of treatments, a continuing need exists for additional treatment options.

### SUMMARY OF THE INVENTION

Embodiments of the invention disclosed herein are directed to the use of effective combinations of TuAAs for the immunotherapy of patients with various types of cancer. Both immunogenic compositions for inducing an immune response to these combinations of antigens and methods for their use are disclosed.

Some embodiments relate to methods of treating neoplastic diseases. The methods can include the step of immunizing a patient against, for example, PRAME and at least one other tumor associated antigen. To immunize a patient against an antigen such as, for example, PRAME, means to administer to the patient some portion of the antigen, or some other immunogenic agent, that is capable of inducing a specific immune response directed to the antigen. Accordingly, in some embodiments, immunizing against PRAME can include administering a complete and intact PRAME antigen to the patient, while in other embodiments it can include administering one or more epitopes, one or more epitope clusters, one or more fragments, and the like, of PRAME, and/or administering, for example, a nucleic acid encoding any of the foregoing epitope(s), cluster(s), fragment(s), and the like. Although PRAME is used in an exemplary way for convenience in this discussion, the principle is applicable to any antigen against which a patient may be immunized.

One embodiment relates to methods of treating ovarian or colorectal cancer. The methods can include the step of immunizing a patient against, for example, PRAME and at least one other tumor associated antigen. In some embodiments the tumor associated antigen can include, for example, SSX-2, NY-ESO-1, PSMA, and the like. Preferably, the methods can include immunizing against PRAME, NY-ESO-1 and SSX-2, for example. More preferably, the methods can include immunization against PRAME, NY-ESO-1, SSX-2, and PSMA, for example. The method can further include the step of immunizing against at least one antigen associated with tumor neovasculature. The antigen associated with tumor neovasculature can be, for example, PSMA, VEGFR2, and Tie-2. In a preferred embodiment, the methods can induce a cytolytic T cell response.

In one embodiment, a method of inducing a cytolytic T-cell response in the treatment of ovarian or colorectal cancer is disclosed. The method can include, for example, the step of immunizing a patient against PRAME and at least one of the tumor-associated antigens. In some embodiments, the tumor-associated antigen can be, for example, SSX-2, NY-ESO-1, PSMA, and the like. For example, the method can include immunization against PRAME, NY-ESO-1, and SSX-2. The method can further include immunization against PSMA. The method can further include the step of immunizing against at least one antigen associated with tumor neovasculature. The antigen associated with tumor neovasculature can be, for example, PSMA, VEGFR2, and Tie-2.

Other embodiments relate to methods of treating pancreatic cancer. The methods can include the step of immunizing against, for example, PSMA and at least one other tumor-associated antigen. The tumor-associated antigen can be, for example, SSX-2 and NY-ESO-1. Preferably, the methods can include immunizing against PSMA, NY-ESO-1, and SSX-2. The method can further include the step of immunizing against at least one antigen associated with tumor neovasculature. The antigen associated with tumor neovasculature can be, for example, VEGFR2 and Tie-2. In a preferred embodiment, the methods can induce a cytolytic T cell response.

Further, in one embodiment, a method of inducing a cytolytic T-cell response in the treatment of pancreatic cancer is disclosed. The method can include, for example, the step of immunizing a patient against PSMA and at least one of the tumor-associated antigens. In some embodiments, the tumor-associated antigen can be, for example, SSX-2, NY-ESO-1, and the like. For example, the method can include immunization against PSMA, NY-ESO-1, and SSX-2. The method can further include the step of immunizing against at least one antigen associated with tumor neovasculature. The antigen associated with tumor neovasculature can be, for example, VEGFR2 and Tie-2.

Still other embodiments relate to methods of treating non-small cell lung cancer. The methods can include the step of immunizing a patient against, for example, PSMA and at least one other tumor associated antigen. For example, the tumor-associated antigen can be MAGE-3, SSX-2, NY-ESO-1, and the like. Preferably, the methods can include immunizing against PSMA, NY-ESO-1 and SSX-2. More preferably, the methods can include immunization against PSMA, NY-ESO-1, SSX-2, and MAGE-3, for example. The method can further include the step of immunizing against at least one antigen associated with tumor neovasculature. The antigen associated with tumor neovasculature can be, for example, VEGFR2 and Tie-2. In a preferred embodiment, the methods can induce a cytolytic T cell response.

In a further embodiment, a method of inducing a cytolytic T-cell response in the treatment of non-small cell lung cancer is disclosed. The method can include, for example, the step of immunizing a patient against PSMA and at least one of tumor-associated antigen. The tumor-associated antigen can be, for example, MAGE-3, SSX-2, NY-ESO-1, and the like. For example, the method can include immunization against PSMA, NY-ESO-1, and SSX-2. The method can further include immunization against PSMA, NY-ESO-1, SSX-2, and MAGE-3. The method can further include the step of immunizing against at least one antigen associated with tumor neovasculature. The antigen associated with tumor neovasculature can be, for example, VEGFR2 and Tie-2.

Alternative embodiments relate to methods of treating renal cell carcinoma. The methods can include the step of immunizing a patient against, for example, PSMA and at least one other tumor associated antigen. The tumor-associated antigen can be, for example, PRAME, SSX-2, and the like. Preferably, the methods can include immunizing against PSMA, PRAME, and SSX-2, for example. The method can further include the step of immunizing against at least one antigen associated with tumor neovasculature. The antigen associated with tumor neovasculature can be, for example, VEGFR2 and Tie-2. In a preferred embodiment, the methods can induce a cytolytic T cell response.

Further, in one embodiment, a method of inducing a cytolytic T-cell response in the treatment of renal cell carcinoma is disclosed. The method can include, for example, the step of immunizing a patient against PSMA and at least one other tumor-associated antigen. In some embodiments, the tumor-associated antigen can be PRAME, SSX-2, and the like. For example, the method can include immunization against PSMA, PRAME, and SSX-2. The method can further include the step of immunizing against at least one antigen associated with tumor neovasculature. The antigen associated with tumor neovasculature can be, for example, VEGFR2 and Tie-2.

Some embodiments relate to methods of treating melanoma. The methods can include the step of immunizing a patient against, for example, at least one tumor-associated antigen selected from each of two groups. The first group can include, for example, tyrosinase, Melan-A, and the like. The second group can include, for example, SSX-2, NY-ESO-1, and the like. Preferably, the method can include immunizing against Melan-A, SSX-2, and NY-ESO. More preferably, the method can include immunization against Melan-A, SSX-2, and tyrosinase, for example. The method can further include the step of immunizing against at least one antigen associated with tumor neovasculature. The antigen associated with tumor neovasculature can be, for example, PSMA, VEGFR2, and Tie-2. In a preferred embodiment, the methods can induce a cytolytic T cell response.

In one embodiment, a method of inducing a cytolytic T-cell response in the treatment of melanoma is disclosed. The method can include, for example, the step of immunizing a patient against at least one tumor-associated antigen selected from each of two groups. The first group can include, for example, tyrosinase, Melan-A, and the like. The second group can include, for example, SSX-2, NY-ESO-1, and the like. For example, the method can include immunization against Melan-A, SSX-2, and NY-ESO. Alternatively, the method can include, for example, immunization against Melan-A, SSX-2, and tyrosinase. The method can further include the step of immunizing against at least one antigen associated with tumor neovasculature. The antigen associated with tumor neovasculature can be, for example, PSMA, VEGFR2, and Tie-2.

Some embodiments relate to the use of a composition comprising, for example, PRAME and at least one other tumor associated antigen, in the preparation of a medicament for the treatment of treating ovarian or colorectal cancer. In some embodiments the tumor associated antigen can include, for example, SSX-2, NY-ESO-1, PSMA, and the like. Preferably, the tumor-associated antigens can be NY-ESO-1 and SSX-2, for example. More preferably, the tumor-associated antigens can be NY-ESO-1, SSX-2, and PSMA, for example. The method can further include the step of immunizing against at least one antigen associated with tumor neovasculature. The antigen associated with tumor neovasculature can be, for example, PSMA, VEGFR2, and Tie-2. In a preferred embodiment, the medicament can induce a cytolytic T cell response.

Other embodiments relate to the use of a composition comprising, for example, PSMA and at least one other tumor-associated antigen in the preparation of medicament for the treatment of pancreatic cancer. The tumor-associated antigen can be, for example, SSX-2 and NY-ESO-1. Preferably, the medicament comprises PSMA, NY-ESO-1, and SSX-2. The method can further include the step of immunizing against at least one antigen associated with tumor neovasculature. The antigen associated with tumor neovasculature can be, for example, VEGFR2 and Tie-2. In a preferred embodiment, the medicament can induce a cytolytic T cell response.

Still other embodiments relate to the use of a composition comprising PSMA and at least one other tumor associated antigen in the preparation of a medicament for the treatment of non-small cell lung cancer. In some embodiments, the tumor-associated antigen can be MAGE-3, SSX-2, NY-ESO-1, and the like. Preferably, the composition comprises PSMA, NY-ESO-1 and SSX-2. More preferably, the composition comprises PSMA, NY-ESO-1, SSX-2, and MAGE-3, for example. The method can further include the step of immunizing against at least one antigen associated with tumor neovasculature. The antigen associated with tumor neovasculature can be, for example, VEGFR2 and Tie-2. In a preferred embodiment, the medicament can induce a cytolytic T cell response.

Alternative embodiments relate to the use of a composition comprising PSMA and at least one other tumor associated antigen in the preparation of a medicament for the treatment of renal cell carcinoma. The tumor-associated antigen can be, for example, PRAME, SSX-2, and the like. Preferably, the composition comprises PSMA, PRAME, and SSX-2, for example. The method can further include the step of immunizing against at least one antigen associated with tumor neovasculature. The antigen associated with tumor neovasculature can be, for example, VEGFR2 and Tie-2. In a preferred embodiment, the medicament can induce a cytolytic T cell response.

Still other embodiments relate to the use of a composition comprising at least one tumor-associated antigen selected from each of two groups in the preparation of a medicament for the treatment of melanoma. The first group can include, for example, tyrosinase, Melan-A, and the like. The second group can include, for example, SSX-2, NY-ESO-1, and the like. Preferably, the composition comprises Melan-A, SSX-2, and NY-ESO. More preferably, the composition comprises Melan-A, SSX-2, and tyrosinase, for example. The method can further include the step of immunizing against at least one antigen associated with tumor neovasculature. The antigen associated with tumor neovasculature can be, for example, PSMA, VEGFR2, and Tie-2. In a preferred embodiment, the medicament can induce a cytolytic T cell response.

Other embodiments relate to immunogenic compositions for the treatment of or for inducing a T cell response against ovarian or colorectal cancer, for example. The compositions can include, for example, individually or in combination, 1) whole antigens, 2) fragments of antigens, 3) epitope clusters derived from antigens, 4) epitopes derived from antigens, 5) nucleic acids encoding any of 1 to 4, and the like; wherein the antigens can include PRAME and a tumor-associated antigen, including, for example, SSX-2, NY-ESO-1, PSMA, and the like. The compositions can further include at least one antigen associated with tumor neovasculature. The antigen associated with tumor neovasculature can be, for example, PSMA, VEGFR2 and Tie-2.

Still other embodiments relate to immunogenic compositions for the treatment of or for inducing a T cell response against pancreatic cancer. The compositions can include, for example, individually or in combination, 1) whole antigens, 2) fragments of antigens, 3) epitope clusters derived from antigens, 4) epitopes derived from antigens, or 5) nucleic acids encoding any of 1 to 4; wherein the antigens can include PSMA and at least one tumor-associated antigen. In a preferred embodiment, the tumor-associated antigen can be selected from SSX-2 and NY-ESO-1, for example. The compositions can further include at least one antigen associated with tumor neovasculature. The antigen associated with tumor neovasculature can be, for example, VEGFR2 and Tie-2.

Alternative embodiments relate to immunogenic compositions for the treatment of or for inducing a T cell response against non-small cell lung cancer. The compositions can include, for example, individually or in combination, 1) whole antigens, 2) fragments of antigens, 3) epitope clusters derived from antigens, 4) epitopes derived from antigens, or 5) nucleic acids encoding any of 1 to 4; wherein the antigens can include PSMA and at least one tumor-associated antigen, including, for example, MAGE-3, SSX-2 and NY-ESO-1. The compositions can further include at least one antigen associated with tumor neovasculature. The antigen associated with tumor neovasculature can be, for example, VEGFR2 and Tie-2.

Some embodiments relate to immunogenic compositions for the treatment of or for inducing a T cell response against renal cell carcinoma. The compositions can include, for example, individually or in combination, 1) whole antigens, 2) fragments of antigens, 3) epitope clusters derived from antigens, 4) epitopes derived from antigens, or 5) nucleic acids encoding any of 1 to 4; wherein the antigens can include PSMA and at least one tumor-associated antigen. In a preferred embodiment the tumor associated antigen can be PRAME or SSX-2. The compositions can further include at least one antigen associated with tumor neovasculature. The antigen associated with tumor neovasculature can be, for example, VEGFR2 and Tie-2.

Still other embodiments relate to an immunogenic composition for the treatment of or for inducing a T cell response against melanoma. The composition can include, for example, individually or in combination, 1) whole antigens, 2) fragments of antigens, 3) epitope clusters derived from antigens, 4) epitopes derived from antigens, or 5) nucleic acids encoding any of 1 to 4; wherein the antigens can be selected from each of two groups; wherein the first group includes, for example, tyrosinase and melan-A; and wherein the second group includes SSX-2 and NY-ESO-1, and the like. The compositions can further include at least one antigen associated with tumor neovasculature. The antigen associated with tumor neovasculature can be, for example, PSMA, VEGFR2 and Tie-2.

Further embodiments relate to compositions and methods for inducing a CTL response using combinations of tumor-associated antigens, including fragments of tumor-associated antigens, clusters and epitopes. The compositions can include nucleic acid constructs, for example, a single construct that encodes all of the desired antigens. In other embodiments a single construct encodes a single antigen, while in other embodiments one construct can have a combination of epitopes with similar immunogenicity and another construct can have epitopes with similar immunogenicity.

Still other embodiments relate to methods of designing and preparing immunogenic compositions, which methods can include the steps of determining the presence of one or more antigens on a tumor type, and obtaining the one or more antigens for inclusion in a composition that induces CTL.

### BRIEF DESCRIPTION OF THE DRAWINGS

FIG. 1 shows the schedule of immunization with two plasmid (pCBP expressing SSX2 41-49 and pSEM expressing Melan A).

FIG. 2 is a bar graph that shows CTL activity obtained using the protocol in FIG. 1.

FIG. 3 shows the schedule of immunization of an entrain-and-amplify immunization protocol using plasmids and peptides representing two epitopes.

FIG. 4 is a table showing in vivo clearance of epitope-pulsed cells in mice immunized according to the protocol of FIG. 3.

FIG. 5 shows the preferred immunization protocols for inducing multivalent responses.

### DETAILED DESCRIPTION OF THE PREFERRED EMBODIMENT

The frequency of expression of many tumor-associated antigens (TuAAs) in various types of cancers is known. However, the frequency of appearance of some antigens, and especially certain combinations of TuAAs in various types of cancers has not been reported. Accurate measurement of the presence of TuAAs in tumor tissues aids in determining which TuAAs will be useful for the treatment of a particular type of cancer.

Many attempts to develop active immunotherapies for cancer have utilized a single antigen. This can be problematic for two distinct reasons. Firstly, the expression of any particular TuAA in cancer can be mosaic with the antigen expression ranging from high in some cells within a tumor mass to completely absent in others. Moreover, the TuAA may be expressed in some lesions but not others. By directing an immune response against more than a single antigen, if properly selected, the number of tumor cells that can be recognized can be maximized. Secondly, it has been observed in some cases that tumors lose expression of a TuAA following immunization, giving rise to a resistant population. If the immune response is directed against more than one TuAA it becomes much more difficult for a resistant tumor to arise because it must then simultaneously lose expression of each of the antigens in order to escape. Thus, in treating cancer with immunotherapy, it can be advantageous to use a combination of TuAAs both due to more complete coverage of the population of tumor cells, and because there will be less chance of tumor escape through antigen loss of expression of the TuAAs, provided the tumor is positive for at least two of the TuAAs used.

### DEFINITIONS

Unless otherwise clear from the context of the use of a term herein, the following listed terms shall generally have the indicated meanings for purposes of this description.

PROFESSIONAL ANTIGEN-PRESENTING CELL (pAPC)—a cell that possesses T cell costimulatory molecules and is able to induce a T cell response. Well characterized pAPCs include dendritic cells, B cells, and macrophages.

PERIPHERAL CELL—a cell that is not a pAPC.

HOUSEKEEPING PROTEASOME—a proteasome normally active in peripheral cells, and generally not present or not strongly active in pAPCs.

IMMUNOPROTEASOME—a proteasome normally active in pAPCs; the immunoproteasome is also active in some peripheral cells in infected tissues or following exposure to interferon.

EPITOPE—a molecule or substance capable of stimulating an immune response. In preferred embodiments, epitopes according to this definition include but are not necessarily limited to a polypeptide and a nucleic acid encoding a polypeptide, wherein the polypeptide is capable of stimulating an immune response. In other preferred embodiments, epitopes according to this definition include but are not necessarily limited to peptides presented on the surface of cells, the peptides being non-covalently bound to the binding cleft of class I MHC, such that they can interact with T cell receptors (TCR). Epitopes presented by class I MHC may be in immature or mature form. “Mature” refers to an MHC epitope in distinction to any precursor (“immature”) that may include or consist essentially of a housekeeping epitope, but also includes other sequences in a primary translation product that are removed by processing, including without limitation, alone or in any combination, proteasomal digestion, N-terminal trimming, or the action of exogenous enzymatic activities. Thus, a mature epitope may be provided embedded in a somewhat longer polypeptide, the immunological potential of which is due, at least in part, to the embedded epitope; likewise, the mature epitope can be provided in its ultimate form that can bind in the MHC binding cleft to be recognized by TCR.

MHC EPITOPE—a polypeptide having a known or predicted binding affinity for a mammalian class I or class II major histocompatibility complex (MHC) molecule.

HOUSEKEEPING EPITOPE—In a preferred embodiment, a housekeeping epitope is defined as a polypeptide fragment that is an MHC epitope, and that is displayed on a cell in which housekeeping proteasomes are predominantly active. In another preferred embodiment, a housekeeping epitope is defined as a polypeptide containing a housekeeping epitope according to the foregoing definition, that is flanked by one to several additional amino acids. In another preferred embodiment, a housekeeping epitope is defined as a nucleic acid that encodes a housekeeping epitope according to the foregoing definitions. Exemplary housekeeping epitopes are provided in U.S. application Ser. Nos. 10/117,937, filed on Apr. 4, 2002 (Pub. No. 20030220239 A1), and 10/657,022, and in PCT Application No. PCT/US2003/027706 (Pub. No. WO04022709A2), filed Sep. 5, 2003; and U.S. Provisional Application Nos. 60/282,211, filed on Apr. 6, 2001; 60/337,017, filed on Nov. 7, 2001; 60/363,210 filed Mar. 7, 2002; and 60/409,123, filed on Sep. 5, 2002. Each of the listed applications is entitled EPITOPE SEQUENCES. Each of the applications mentioned in this paragraph is incorporated herein by reference in its entirety.

IMMUNE EPITOPE—In a preferred embodiment, an immune epitope is defined as a polypeptide fragment that is an MHC epitope, and that is displayed on a cell in which immunoproteasomes are predominantly active. In another preferred embodiment, an immune epitope is defined as a polypeptide containing an immune epitope according to the foregoing definition, that is flanked by one to several additional amino acids. In another preferred embodiment, an immune epitope is defined as a polypeptide including an epitope cluster sequence, having at least two polypeptide sequences having a known or predicted affinity for a class I MHC. In yet another preferred embodiment, an immune epitope is defined as a nucleic acid that encodes an immune epitope according to any of the foregoing definitions.

TARGET CELL—In a preferred embodiment, a target cells is a cell associated with a pathogenic condition that can be acted upon by the components of the immune system, for example, a cell infected with a virus or other intracellular parasite, or a neoplastic cell. In another embodiment, a target cell is a cell to be targeted by the vaccines and methods of the invention. Examples of target cells according to this definition include but are not necessarily limited to: a neoplastic cell and a cell harboring an intracellular parasite, such as, for example, a virus, a bacterium, or a protozoan. Target cells can also include cells that are targeted by CTL as a part of an assay to determine or confirm proper epitope liberation and processing by a cell expressing immunoproteasome, to determine T cell specificity or immunogenicity for a desired epitope. Such cells can be transformed to express the liberation sequence, or the cells can simply be pulsed with peptide/epitope.

TARGET-ASSOCIATED ANTIGEN (TAA)—a protein or polypeptide present in a target cell.

TUMOR-ASSOCIATED ANTIGEN (TuAA)—a TAA, wherein the target cell is a neoplastic cell.

HLA EPITOPE—a polypeptide having a known or predicted binding affinity for a human class I or class II HLA complex molecule.

ANTIBODY—a natural immunoglobulin (Ig), poly- or monoclonal, or any molecule composed in whole or in part of an Ig binding domain, whether derived biochemically, or by use of recombinant DNA, or by any other means. Examples include inter alia, F(ab), single chain Fv, and Ig variable region-phage coat protein fusions.

SUBSTANTIAL SIMILARITY—this term is used to refer to sequences that differ from a reference sequence in an inconsequential way as judged by examination of the sequence. Nucleic acid sequences encoding the same amino acid sequence are substantially similar despite differences in degenerate positions or minor differences in length or composition of any non-coding regions. Amino acid sequences differing only by conservative substitution or minor length variations are substantially similar. Additionally, amino acid sequences comprising housekeeping epitopes that differ in the number of N-terminal flanking residues, or immune epitopes and epitope clusters that differ in the number of flanking residues at either terminus, are substantially similar. Nucleic acids that encode substantially similar amino acid sequences are themselves also substantially similar.

FUNCTIONAL SIMILARITY—this term is used to refer to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus may not be within the substantial similarity definition). Pairs of antibodies, or TCRs, that recognize the same epitope can be functionally similar to each other despite whatever structural differences exist. Testing for functional similarity of immunogenicity can be conducted by immunizing with the “altered” antigen and testing the ability of an elicited response, including but not limited to an antibody response, a CTL response, cytokine production, and the like, to recognize the target antigen. Accordingly, two sequences may be designed to differ in certain respects while retaining the same function. Such designed sequence variants of disclosed or claimed sequences are among the embodiments of the present invention.

EXPRESSION CASSETTE—a polynucleotide sequence encoding a polypeptide, operably linked to a promoter and other transcription and translation control elements, including but not limited to enhancers, termination codons, internal ribosome entry sites, and polyadenylation sites. The cassette can also include sequences that facilitate moving it from one host molecule to another.

EMBEDDED EPITOPE—in some embodiments, an embedded epitope is an epitope that is wholly contained within a longer polypeptide; in other embodiments, the term also can include an epitope in which only the N-terminus or the C-terminus is embedded such that the epitope is not wholly in an interior position with respect to the longer polypeptide.

MATURE EPITOPE—a peptide with no additional sequence beyond that present when the epitope is bound in the MHC peptide-binding cleft.

EPITOPE CLUSTER—a polypeptide, or a nucleic acid sequence encoding it, that is a segment of a protein sequence, including a native protein sequence, comprising two or more known or predicted epitopes with binding affinity for a shared MHC restriction element. In preferred embodiments, the density of epitopes within the cluster is greater than the density of all known or predicted epitopes with binding affinity for the shared MHC restriction element within the complete protein sequence. Epitope clusters are disclosed and more fully defined in U.S. patent application Ser. No. 09/561,571 entitled EPITOPE CLUSTERS, which is incorporated herein by reference in its entirety.

LIBERATION SEQUENCE—a designed or engineered sequence comprising or encoding a housekeeping epitope embedded in a larger sequence that provides a context allowing the housekeeping epitope to be liberated by processing activities including, for example, immunoproteasome activity, N terminal trimming, and/or other processes or activities, alone or in any combination.

CTLp—CTL precursors are T cells that can be induced to exhibit cytolytic activity. Secondary in vitro lytic activity, by which CTLp are generally observed, can arise from any combination of naïve, effector, and memory CTL in vivo.

MEMORY T CELL—A T cell, regardless of its location in the body, that has been previously activated by antigen, but is in a quiescent physiologic state requiring re-exposure to antigen in order to gain effector function. Phenotypically they are generally CD62L⁻ CD44^{hi} CD107α⁻ IGN-γ⁻ LTβ⁻ TNF-α⁻ and is in G0 of the cell cycle.

EFFECTOR T CELL—A T cell that, upon encountering antigen, readily exhibits effector function. Effector T cells are generally capable of exiting the lymphatic system and entering the immunological periphery. Phenotypically they are generally CD62L⁻ CD44^{hi} CD107α⁺ IGN-γ⁺ LTβ⁺ TNF-α⁺ and actively cycling.

EFFECTOR FUNCTION—Generally, T cell activation generally, including acquisition of cytolytic activity and/or cytokine secretion.

INDUCING a T cell response—Includes in many embodiments the process of generating a T cell response from naïve, or in some contexts, quiescent cells; activating T cells.

AMPLIFYING a T cell response—Includes in many embodiments the process or increasing the number of cells, the number of activated cells, the level of activity, rate of proliferation, or similar parameter of T cells involved in a specific response.

ENTRAINMENT—Includes in many embodiments an induction that confers particular stability on the immune profile of the induced lineage of T cells.

TOLL-LIKE RECEPTOR (TLR)—Toll-like receptors (TLRs) are a family of pattern recognition receptors that are activated by specific components of microbes and certain host molecules. As part of the innate immune system, they contribute to the first line of defense against many pathogens, but also play a role in adaptive immunity.

TOLL-LIKE RECEPTOR (TLR) LIGAND—Any molecule capable of binding and activating a toll-like receptor. Examples include, without limitation: poly IC A synthetic, double-stranded RNA know for inducing interferon. The polymer is made of one strand each of polyinosinic acid and polycytidylic acid, double-stranded RNA, unmethylated CpG oligodeoxyribonucleotide or other immunostimulatory sequences (ISSs), lipopolysacharide (LPS), β-glucans, and imidazoquinolines, as well as derivatives and analogues thereof.

IMMUNOPOTENTIATING ADJUVANTS—Adjuvants that activate pAPC or T cells including, for example: TLR ligands, endocytic-Pattern Recognition Receptor (PRR) ligands, quillaja saponins, tucaresol, cytokines, and the like. Some preferred adjuvants are disclosed in Marciani, D. J. Drug Discovery Today 8:934-943, 2003, which is incorporated herein by reference in its entirety.

IMNUNOSTIMULATORY SEQUENCE (ISS)—Generally an oligodeoxyribonucleotide containing an unmethlylated CpG sequence. The CpG may also be embedded in bacterially produced DNA, particularly plasmids. Further embodiments include various analogues; among preferred embodiments are molecules with one or more phosphorothioate bonds or non-physiologic bases.

VACCINE—In preferred embodiments a vaccine can be an immunogenic composition providing or aiding in prevention of disease. In other embodiments, a vaccine is a composition that can provide or aid in a cure of a disease. In others, a vaccine composition can provide or aid in amelioration of a disease. Further embodiments of a vaccine immunogenic composition can be used as therapeutic and/or prophylactic agents.

IMMUNIZATION—a process to induce partial or complete protection against a disease. Alternatively, a process to induce or amplify an immune system response to an antigen. In the second definition it can connote a protective immune response, particularly proinflammatory or active immunity, but can also include a regulatory response. Thus in some embodiments immunization is distinguished from tolerization (a process by which the immune system avoids producing proinflammatory or active immunity) while in other embodiments this term includes tolerization.

ENCODE—an open-ended term such that a nucleic acid encoding a particular amino acid sequence can consist of codons specifying that (poly)peptide, but can also comprise additional sequences either translatable, or for the control of transcription, translation, or replication, or to facilitate manipulation of some host nucleic acid construct.

COVERAGE—the fraction or proportion of tumor cells expressing a particular TuAA or at least one TuAA from a set of selected TuAAs.

REDUNDANCY—the degree to which a population of tumor cells, or some subset of them, express more than one of a selected set of TuAAs.

### Tumor Associated Antigens

Examples of TuAAs useful in embodiments disclosed herein include tyrosinase (SEQ. ID NO. 1), melan-A, (SEQ. ID NO. 2), SSX-2, (SEQ. ID NO. 3), PSMA (prostate-specific membrane antigen) (SEQ. ID NO. 4), MAGE-1, (SEQ. ID NO. 5), MAGE-(SEQ. ID NO. 6), NY-ESO-1, (SEQ. ID NO. 7), PRAME, (SEQ. ID NO. 8), and Her2/Neu (SEQ. ID NO. 9). The natural coding sequences for these nine proteins, or any segments within them, can be determined from their cDNA or complete coding (cds) sequences, SEQ. ID NOS. 10-18, respectively.

TABLE 1
SEQ. ID NOS.

| | | ACCESSION |
|---|---|---|
| SEQ. ID NO. | IDENTITY | NUMBER** |
| 1 | Tyrosinase protein | P14679 |
| 2 | Melan-A protein | Q16655 |
| 3 | SSX-2 protein | NP_003138 |
| 4 | PSMA protein | NP_004467 |
| 5 | MAGE-1 protein | P43355 |
| 6 | MAGE-3 protein | P43357 |
| 7 | NY-ESO-1 protein | P78358 |
| 8 | PRAME protein | NP 006106 |
| 9 | Her2/Neu protein | P04626 |
| 10 | Tyrosinase cDNA | NM_000372 |
| 11 | Melan-A cDNA | U06452 |
| 12 | SSX-2 cDNA | NM_003147 |
| 13 | PSMA cDNA | NM_004476 |
| 14 | MAGE-1 cds | M77481 |
| 15 | MAGE-3 cds | U03735 |
| 16 | NY-ESO-1 cDNA | U87459 |
| 17 | PRAME cDNA | NM_006115 |
| 18 | Her2/Neu cDNA | M11730 |

**All accession numbers used here and throughout can be accessed through the NCBI databases, for example, through the Entrez seek and retrieval system on the world wide web.

Tyrosinase is a melanin biosynthetic enzyme that is considered one of the most specific markers of melanocytic differentiation. Tyrosinase is expressed in few cell types, primarily in melanocytes, and high levels are often found in melanomas. The usefulness of tyrosinase as a TuAA is taught in U.S. Pat. No. 5,747,271 entitled “METHOD FOR IDENTIFYING INDIVIDUALS SUFFERING FROM A CELLULAR ABNORMALITY SOME OF WHOSE ABNORMAL CELLS PRESENT COMPLEXES OF HLA-A2/TYROSINASE DERIVED PEPTIDES, AND METHODS FOR TREATING SAID INDIVIDUALS” which is hereby incorporated by reference in its entirety.

GP100, also known as PMel17, also is a melanin biosynthetic protein expressed at high levels in melanomas. GP100 as a TuAA is disclosed in U.S. Pat. No. 5,844,075 entitled “MELANOMA ANTIGENS AND THEIR USE IN DIAGNOSTIC AND THERAPEUTIC METHODS,” which is hereby incorporated by reference in its entirety.

Melan-A, also called MART-1 (Melanoma Antigen Recognized by T cells), is another melanin biosynthetic protein expressed at high levels in melanomas. The usefulness of Melan-A/MART-1 as a TuAA is taught in U.S. Pat. Nos. 5,874,560 and 5,994,523 both entitled “MELANOMA ANTIGENS AND THEIR USE IN DIAGNOSTIC AND THERAPEUTIC METHODS,” as well as U.S. Pat. No. 5,620,886, entitled “ISOLATED NUCLEIC ACID SEQUENCE CODING FOR A TUMOR REJECTION ANTIGEN PRECURSOR PROCESSED TO AT LEAST ONE TUMOR REJECTION ANTIGEN PRESENTED BY HLA-A2”, each of which is hereby incorporated by reference in its entirety.

SSX-2, also know as Hom-Mel-40, is a member of a family of highly conserved cancer-testis antigens (Gure, A. O. et al. Int. J. Cancer 72:965-971, 1997, which is hereby incorporated by reference in its entirety). Its identification as a TuAA is taught in U.S. Pat. No. 6,025,191 entitled “ISOLATED NUCLEIC ACID MOLECULES WHICH ENCODE A MELANOMA SPECIFIC ANTIGEN AND USES THEREOF,” which is hereby incorporated by reference in its entirety. Cancer-testis antigens are found in a variety of tumors, but are generally absent from normal adult tissues except testis. Expression of different members of the SSX family has been found in various tumor cell lines. Due to the high degree of sequence identity among SSX family members, similar epitopes from more than one member of the family will be generated and able to bind to an MHC molecule, so that some vaccines directed against one member of this family can cross-react and be effective against other members of this family.

MAGE-1 (melanoma-associated antigen-1), MAGE-2 (melanoma-associated antigen-2), and MAGE-3 (melanoma-associated antigen-3) are members of another family of cancer-testis antigens originally discovered in melanoma but found in a variety of tumors. The identification of MAGE proteins as TuAAs is taught in U.S. Pat. No. 5,342,774 entitled NUCLEOTIDE SEQUENCE ENCODING THE TUMOR REJECTION ANTIGEN PRECURSOR, MAGE-1, which is hereby incorporated by reference in its entirety, and in numerous subsequent patents. Currently there are 17 entries for (human) MAGE in the SWISS Protein database. There is extensive similarity among these proteins, such that in many cases, an epitope from one can induce a cross-reactive response to other members of the family. A few members of the MAGE family have not been observed in tumors, most notably MAGE-H1 and MAGE-D1, which are expressed in testes and brain, and bone marrow stromal cells, respectively. The possibility of cross-reactivity on normal tissue is ameliorated by the fact that they are among the least similar to the other MAGE proteins.

GAGE-1 is a member of the GAGE family of cancer testis antigens (Van den Eynde, B., et al., J. Exp. Med. 182: 689-698, 1995; U.S. Pat. Nos. 5,610,013; 5,648,226; 5,858,689; 6,013,481; and 6,069,001). The PubGene database currently lists 12 distinct accessible members, some of which are synonymously known as PAGE or XAGE. GAGE-1 through GAGE-8 have a very high degree of sequence identity, so most epitopes can be shared among multiple members of the family.

BAGE is a cancer-testis antigen commonly expressed in melanoma, particularly metastatic melanoma, as well as in carcinomas of the lung, breast, bladder, and squamous cells of the head and neck. Its usefulness as a TuAA is taught in U.S. Pat. Nos. 5,683,88 entitled “TUMOR REJECTION ANTIGENS WHICH CORRESPOND TO AMINO ACID SEQUENCES IN TUMOR REJECTION ANTIGEN PRECURSOR BAGE, AND USES THEREOF” and 5,571,711 entitled “ISOLATED NUCLEIC ACID MOLECULES CODING FOR BAGE TUMOR REJECTION ANTIGEN PRECURSORS,” each of which is hereby incorporated by reference in its entirety.

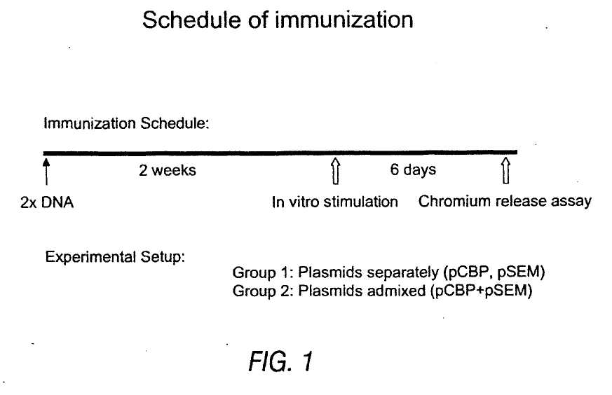

NY-ESO-1, also known as CTAG-1 (Cancer-Testis Antigen-1) and CAG-3 (Cancer Antigen-3), is a cancer-testis antigen found in a wide variety of tumors. NY-ESO-1 as a TuAA is disclosed in U.S. Pat. No. 5,804,381 entitled ISOLATED NUCLEIC ACID MOLECULE ENCODING AN ESOPHAGEAL CANCER ASSOCIATED ANTIGEN, THE ANTIGEN ITSELF, AND USES THEREOF which is hereby incorporated by reference in its entirety. A paralogous locus encoding antigens with extensive sequence identity, LAGE-1a/s and LAGE-1b/L, has been disclosed in publicly available assemblies of the human genome, and has been concluded to arise through alternate splicing. Additionally, CT-2 (or CTAG-2, Cancer-Testis Antigen-2) appears to be either an allele, a mutant, or a sequencing discrepancy of LAGE-1b/L. Due to the extensive sequence identity, many epitopes from NY-ESO-1 can also induce immunity to tumors expressing these other antigens. See FIG. 1. NY-ESO-1 and LAGE are virtually identical through amino acid 70. From amino acid 71 through 134 the longest run of identity between the two proteins is 6 residues, but potentially cross-reactive sequences are present. From amino acid 135 through 180, NY-ESO and LAGE-1a/s are identical except for a single residue, but LAGE-1b/L is unrelated due to the alternate splice. The CAMEL and LAGE-2 antigens appear to derive from the LAGE-1 mRNA, but from alternate reading frames, thus giving rise to unrelated protein sequences. More recently, GenBank Accession AF277315.5, Homo sapiens chromosome X clone RP5-865E18, RP5-1087L19, complete sequence, reports three independent loci in this region which are labeled as LAGE1 (corresponding to CTAG-2 in the genome assemblies), LAGE2-A and LAGE2-B (both corresponding to CTAG-1 in the genome assemblies).

PRAME, also know as MAPE, DAGE, and OIP4, was originally observed as a melanoma antigen. Subsequently, it has been recognized as a cancer-testis (CT) antigen, but unlike many CT antigens, such as, MAGE, GAGE and BAGE, PRAME is expressed in acute myeloid leukemias. PRAME is a member of the MAPE family, which consists largely of hypothetical proteins with which it shares limited sequence similarity. The usefulness of PRAME as a TuAA is taught in U.S. Pat. No. 5,830,753 entitled “ISOLATED NUCLEIC ACID MOLECULES CODING FOR TUMOR REJECTION ANTIGEN PRECURSOR DAGE AND USES THEREOF,” which is hereby incorporated by reference in its entirety.

PSMA (prostate-specific membranes antigen), a TuAA described in U.S. Pat. No. 5,538,866 entitled “PROSTATE-SPECIFIC MEMBRANES ANTIGEN” which is hereby incorporated by reference in its entirety, is expressed by normal prostate epithelium and, at a higher level, in prostatic cancer. It has also been found in the neovasculature of non-prostatic tumors. PSMA can thus form the basis for vaccines directed to both prostate cancer and to the neovasculature of other tumors. This later concept is more fully described in a provisional U.S. Patent Application No. 60/274,063 entitled “ANTI-NEOVASCULAR VACCINES FOR CANCER,” filed Mar. 7, 2001, and U.S. application Ser. No. 10/094,699 (Pub. No. 20030046714 A1), filed on Mar. 7, 2002, entitled “ANTI-NEOVASCULAR PREPARATIONS FOR CANCER,” each of which is hereby incorporated by reference in its entirety. Briefly, as tumors grow they recruit ingrowth of new blood vessels. This is understood to be necessary to sustain growth as the centers of unvascularized tumors are generally necrotic and angiogenesis inhibitors have been reported to cause tumor regression. Such new blood vessels, or neovasculature, express antigens not found in established vessels, and thus can be specifically targeted. By inducing CTL against neovascular antigens the vessels can be disrupted, interrupting the flow of nutrients to, and removal of wastes from, tumors, leading to regression.

Alternate splicing of the PSMA mRNA leads to a protein with an apparent start at Met₅₈, thereby deleting the putative membrane anchor region of PSMA as described in U.S. Pat. No. 5,935,818 entitled “ISOLATED NUCLEIC ACID MOLECULE ENCODING ALTERNATIVELY SPLICED PROSTATE-SPECIFIC MEMBRANES ANTIGEN AND USES THEREOF,” which is hereby incorporated by reference in its entirety. A protein termed PSMA-like protein, Genbank accession number AF261715, is nearly identical to amino acids 309-750 of PSMA, but has a different expression profile. Thus the most preferred epitopes are those with an N-terminus located from amino acid 58 to 308.

PSA, prostate specific antigen, is a peptidase of the kallikrein family and a differentiation antigen of the prostate. Expression in breast tissue has also been reported. Alternate names include gamma-seminoprotein, kallikrein 3, seminogelase, seminin, and P-30 antigen. PSA has a high degree of sequence identity with the various alternate splicing products prostatic/glandular kallikrein-1 and -2, as well as kallikrein 4, which is also expressed in prostate and breast tissue. Other kallikreins generally share less sequence identity and have different expression profiles. Nonetheless, cross-reactivity that might be provoked by any particular epitope, along with the likelihood that that epitope would be liberated by processing in non-target tissues (most generally by the housekeeping proteasome), should be considered in designing a vaccine.

PSCA, prostate stem cell antigen, and also known as SCAH-2, is a differentiation antigen preferentially expressed in prostate epithelial cells, and overexpressed in prostate cancers. Lower level expression is seen in some normal tissues including neuroendocrine cells of the digestive tract and collecting ducts of the kidney. PSCA is described in U.S. Pat. No. 5,856,136 entitled “HUMAN STEM CELL ANTIGENS,” which is hereby incorporated by reference in its entirety.

Synaptonemal complex protein 1 (SCP-1), also known as HOM-TES-14, is a meiosis-associated protein and also a cancer-testis antigen (Tureci, O., et al. Proc. Natl. Acad. Sci. USA 95:5211-5216, 1998). As a cancer antigen its expression is not cell-cycle regulated and it is found frequently in gliomas, breast, renal cell, and ovarian carcinomas. It has some similarity to myosins, but with few enough identities that cross-reactive epitopes are not an immediate prospect.

The ED-B domain of fibronectin is also a potential target. Fibronectin is subject to developmentally regulated alternative splicing, with the ED-B domain being encoded by a single exon that is used primarily in oncofetal tissues (Matsuura, H. and S. Hakomori Proc. Natl. Acad. Sci, USA 82:6517-6521, 1985; Carnemolla, B. et al. J. Cell Biol. 108:1139-1148, 1989; Loridon-Rosa, B. et al. Cancer Res. 50:1608-1612, 1990; Nicolo, G. et al. Cell Differ. Dev. 32:401-408, 1990; Borsi, L. et al. Exp. Cell Res. 199:98-105, 1992; Oyama, F. et al. Cancer Res. 53:2005-2011, 1993; Mandel, U. et al. APMIS 102:695-702, 1994; Farnoud, M. R. et al. Int. J. Cancer 61:27-34, 1995; Pujuguet, P. et al. Am. J. Pathol. 148:579-592, 1996; Gabler, U. et al. Heart 75:358-362, 1996; Chevalier, X. Br. J. Rheumatol. 35:407-415, 1996; Midulla, M. Cancer Res. 60:164-169, 2000).

The ED-B domain is also expressed in fibronectin of the neovasculature (Kaczmarek, J. et al. Int. J. Cancer 59:11-16, 1994; Castellani, P. et al. Int. J. Cancer 59:612-618, 1994; Neri, D. et al. Nat. Biotech. 15:1271-1275, 1997; Karelina, T. V. and A. Z. Eisen Cancer Detect. Prev. 22:438-444, 1998; Tarli, L. et al. Blood 94:192-198, 1999; Castellani, P. et al. Acta Neurochir. (Wien) 142:277-282, 2000). As an oncofetal domain, the ED-B domain is commonly found in the fibronectin expressed by neoplastic cells in addition to being expressed by the neovasculature. Thus, CTL-inducing vaccines targeting the ED-B domain can exhibit two mechanisms of action: direct lysis of tumor cells, and disruption of the tumor's blood supply through destruction of the tumor-associated neovasculature. As CTL activity can decay rapidly after withdrawal of vaccine, interference with normal angiogenesis can be minimal. The design and testing of vaccines targeted to neovasculature is described in Provisional U.S. Patent Application No. 60/274,063 entitled “ANTI-NEOVASCULATURE VACCINES FOR CANCER,” filed on Mar. 7, 2001, and in U.S. patent application Ser. No. 10/094,699, (Pub. No. 20030046714 A1), entitled “ANTI-NEOVASCULATURE PREPARATIONS FOR CANCER,” filed on Mar. 7, 2002, each of which is hereby incorporated by reference in its entirety. A tumor cell line is disclosed in Provisional U.S. Application No. 60/363,131, filed on Mar. 7, 2002, entitled “HLA-TRANSGENIC MURINE TUMOR CELL LINE,” which is hereby incorporated by reference in its entirety.

Carcinoembryonic antigen (CEA) is a paradigmatic oncofetal protein first described in 1965 (Gold and Freedman, J. Exp. Med. 121: 439-462, 1965. Fuller references can be found in the Online Medelian Inheritance in Man; record *114890. It has officially been renamed carcinoembryonic antigen-related cell adhesion molecule 5 (CEACAM5). Its expression is most strongly associated with adenocarcinomas of the epithelial lining of the digestive tract and in fetal colon. CEA is a member of the immunoglobulin supergene family and the defining member of the CEA subfamily.

Survivin, also known as Baculoviral IAP Repeat-Containing Protein 5 (BIRC5), is another protein with an oncofetal pattern of expression. It is a member of the inhibitor of apoptosis protein (IAP) gene family. It is widely over-expressed in cancers (Ambrosini, G. et al., Nat. Med. 3:917-921, 1997; Velculiscu V. E. et al., Nat. Genet. 23:387-388, 1999) and its function as an inhibitor of apoptosis is believed to contribute to the malignant phenotype.

HER2/NEU is an oncogene related to the epidermal growth factor receptor (van de Vijver, et al., New Eng. J. Med. 319:1239-1245, 1988), and apparently identical to the c-ERBB2 oncogene (Di Fiore, et al., Science 237: 178-182, 1987). The over-expression of ERBB2 has been implicated in the neoplastic transformation of prostate cancer. As with HER2, it is amplified and over-expressed in 25-30% of breast cancers among other tumors where expression level is correlated with the aggressiveness of the tumor (Slamon, et al., New Eng. J. Med. 344:783-792, 2001). A more detailed description is available in the Online Medelian Inheritance in Man; record *164870.

Further examples of tumor-associated antigens include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

Additional tumor-associated antigens are described in Chen, Y T, “Identification of human tumor antigens by serological expression cloning: an online review on SEREX” Cancer Immun. 2004 [updated 2004 Mar. 10; cited 2004 Apr. 1] at world wide web cancerimmunotherapy.org/SEREX/; and Renkvist, N. et al., “A listing of tumor antigens recognized by T cells,” Cancer Immunology Immunotherapy, 50:3-15 (2001), each of which is hereby incorporated by reference in its entirety.

Table 2, adapted from Scanlan et al., “The cancer/testis genes: Review, standardization, and commentary,” Cancer Immunity 4:1 (Jan. 23, 2004), which is hereby incorporated by reference in its entirety, provides a listing of CT Antigens. Table 3 provides the frequency of mRNA expression in various tumor types for the CT antigens in Table 2. Scanlan et al., “The cancer/testis genes: Review, standardization, and commentary,” Cancer Immunity 4:1 (Jan. 23, 2004), which is hereby incorporated by reference in its entirety.

TABLE 2
Listing of CT genes

| CT | Transcript/Transcript | |
|---|---|---|
| Identifier | family | Family Members/CT Identifier (Synonyms) |
| CT1 | MAGEA | MAGEA1/CT1.1, MAGEA2/CT1.2, |
| | | MAGEA3/CT1.3, MAGEA4/CT1.4, |
| | | MAGEA5/CT1.5, MAGEA6/CT1.6, |
| | | MAGEA7/CT1.7, MAGEA8/CT1.8, |
| | | MAGEA9/CT.9, MAGEA10/CT1.10, |
| | | MAGEA11/CT1.11, MAGEA12/CT1.12 |
| CT2 | BAGE | BAGE/CT2.1, BAGE2/CT2.2, BAGE3/CT2.3, |
| | | BAGE4/CT2.4, BAGE5/CT2.5 |
| CT3 | MAGEB | MAGEB1/CT3.1, MAGEB2/CT3.2, |
| | | MAGEB5/CT3.3, MAGEB6/CT3.4 |
| CT4 | GAGE1 | GAGE1/CT4.1, GAGE2/CT4.2, GAGE3/CT4.3, |
| | | GAGE4/CT4.4, GAGE5/CT4.5, GAGE6/CT4.6, |
| | | GAGE7/CT4.7, GAGE8/CT4.8 |
| CT5 | SSX | SSX1/CT5.1, SSX2/CT5.2a, SSX2/CT5.2b, |
| | | SSX3/CT5.3, SSX4/CT5.4 |
| CT6 | NY-ESO-1 | NY-ESO-1/CT6.1, LAGE-1a/CT6.2a, LAGE- |
| | | 1b/CT6.2b |
| CT7 | MAGEC1 | MAGEC1/CT7.1, MAGEC3/CT7.2 |
| CT8 | SYCP1 | SYCP1/CT8 |
| CT9 | BRDT | BRDT/CT9 |
| CT10 | MAGEE1 | MAGEE1/CT10 |
| CT11 | CTp11/SPANX | SPANXA1/CT11.1, SPANXB1/CT11.2, |
| | | SPANXC/CT11.3, SPANXD/CT11.4 |
| CT12 | XAGE-1/GAGED | XAGE-1a/CT12.1a, XAGE-1b/CT12.1b, XAGE- |
| | | 1c/CT12.1c, XAGE-1d/CT12.1d, XAGE-2/CT12.2, |
| | | XAGE-3a/CT12.3a, XAGE-3b/CT12.3b, XAGE- |
| | | 4/CT12.4 |
| CT13 | HAGE | HAGE/CT13 |
| CT14 | SAGE | SAGE/CT14 |
| CT15 | ADAM2 | ADAM2/CT15 |
| CT16 | PAGE-5 | PAGE-5/CT16.1, CT16.2 |
| CT17 | LIP1 | LIP1/CT17 |
| CT18 | NA88 | NA88/CT12 |
| CT19 | IL13RA1 | IL13RA1/CT19 |
| CT20 | TSP50 | TSP50/CT20 |
| CT21 | CTAGE-1 | CTAGE-1/CT21.1, CTAGE-2/CT21.2 |
| CT22 | SPA17 | SPA17/CT22 |
| CT23 | OY-TES-1 | OY-TES-1/CT23 |
| CT24 | CSAGE | CSAGE/CT24.1, TRAG3/CT24.2 |
| CT25 | MMA1/DSCR8 | MMA-1a/CT25.1a, MMA-1b/CT25.1b |
| CT26 | CAGE | CAGE/CT26 |
| CT27 | BORIS | BORIS/CT27 |
| CT28 | HOM-TES-85 | HOM-TES-85/CT28 |
| CT29 | AF15q14/D40 | D40/CT29 |
| CT30 | E2F-like/HCA661 | HCA661/CT30 |
| CT31 | PLU-1 | PLU-1/CT31 |
| CT32 | LDHC | LDHC/CT32 |
| CT33 | MORC | MORC/CT33 |
| CT34 | SGY-1 | SGY-1/CT34 |
| CT35 | SPO11 | SPO11/CT35 |
| CT36 | TPX1 | TPX-1/CT36 |
| CT37 | NY-SAR-35 | NY-SAR-35/CT37 |
| CT38 | FTHL17 | FTHL17/CT38 |
| CT39 | NXF2 | NXF2/CT39 |
| CT40 | TAF7L | TAF7L/CT40 |
| CT41 | TDRD1 | TDRD1/CT41.1, NY-CO-45/CT41.2 |
| CT42 | TEX15 | TEX15/CT42 |
| CT43 | FATE | FATE/CT43 |
| CT44 | TPTE | TPTE/CT44 |
| — | PRAME | (MAPE, DAGE) |

TABLE 3

| | Frequency (%) of Expression in Tumor Type | | | | | | | | | | | | | | | | |
|---|---|---|---|---|---|---|---|---|---|---|---|---|---|---|---|---|---|
| CT Family | | | | | | | | | Leuk/ | Lung | | | | | | | |
| (Member) | Blad | Brn | Brst | Col | Eso | Gas | H/N | Liver | Lymph | (NSCLC) | Mel | Ov | Pancr | Pros | Renal | Sarc | Ref |
| MAGEA1/CT1.1 | 22 | — | 18 | 2 | 53 | 29 | 28 | 80 | 0 | 49 | 48 | 28 | — | 15 | 0 | 14 | 44 |
| BAGE1/CT2.1 | 15 | — | 10 | 0 | — | — | 8 | — | 0 | 4 | 26 | 15 | — | 0 | 0 | 6 | 44 |
| MAGEB1/CT3.1 | 0 | 0 | 17 | 0 | — | 0 | 0 | — | 0 | 14 | 22 | — | — | 0 | 0 | 9 | 45 |
| GAGE/CT4.1 | 12 | — | 9 | 0 | — | — | 19 | 38^{b} | 1 | 19 | 28 | 31 | — | 10 | 0 | 25 | 44 |
| SSX2/CT5.2 | 44 | 6 | 7 | 12 | — | — | 35 | 9^{b} | 36 | 16 | 35 | — | — | 40 | 5 | 50 | 46 |
| NY-ESO-1/CT6.1 | 80 | 0 | 30 | 0 | — | 0 | — | 29 | 0 | 17 | 34 | 25 | 0 | 25 | 9 | 0 | 8 |
| MAGEC1/CT7.1 | 44 | — | 30 | 10 | — | — | 36 | — | — | 33 | 70 | — | — | — | — | 60 | 20 |
| SYCP1/CT8 | — | 47 | 20 | 0 | — | 7 | — | 28^{b} | 0 | 7 | 14 | 0 | — | 0 | 8 | 0 | 9 |
| BRDT/CT9 | 0 | — | 0 | 0 | 8 | — | 8 | — | — | 25 | 0 | — | — | — | 0 | — | 16 |
| MAGEE1/CT10 | 44 | — | 38 | 0 | — | — | 36 | — | — | 24 | 50 | — | — | — | — | 0 | 12 |
| SPANXC/CT11.3 | 9 | — | 25 | 22 | 0 | — | — | — | — | 33 | 70 | — | 0 | — | — | — | 14 |
| XAGE-1a/CT12.1a | — | — | — | — | — | — | — | — | — | — | 8 | — | — | — | — | 22 | 47 |
| HAGE/CT13 | 24 | 37 | 5 | 31 | 27 | — | — | 20 | 9 | 32 | 17 | — | — | 22 | 6 | 20 | 13 |
| SAGE/CT14 | 12 | 0 | 5 | 0 | 20 | — | 17 | — | 4 | 22 | 4 | — | — | 0 | 5 | 5 | 13 |
| ADAM2/CT15 | — | — | 0 | 0 | — | — | — | — | — | 0 | 0 | 0 | — | — | 12 | — | 17 |
| PAGE-5/CT16 | — | — | 5 | 11 | — | — | — | — | — | 39 | 22 | 0 | — | — | 44 | — | 17 |
| LIP1/CT17 | — | — | 5 | 0 | — | — | — | — | — | 0 | 0 | 0 | — | — | 25 | — | 17 |
| NA88/CT18 | — | — | — | — | — | — | — | — | — | — | 11 | — | — | — | — | — | 48 |
| TSP50/CT20 | — | — | 28 | — | — | — | — | — | — | — | — | — | — | — | — | — | 49 |
| CTAGE-1/CT21.1 | — | — | — | — | — | — | — | — | 35 | — | — | — | — | — | — | — | 50 |
| SPA17/CT22 | — | — | — | — | — | — | — | — | 26 | — | — | — | — | — | — | — | 51 |
| OYTES1/CT23 | 28 | — | 40 | 15 | — | 0 | — | 40 | — | 20 | — | — | — | — | 0 | — | 52 |
| MMA1a/CT25.1a | — | — | 0 | 0 | 0 | — | — | — | — | 40 | 26 | — | 0 | — | — | 18 | 15 |
| CAGE/CT26 | — | — | — | — | — | 89 | — | — | — | 100 | — | — | — | — | — | — | 53 |
| HOMTES85/CT28 | — | 35 | 0 | 10 | — | — | — | 19 | — | 28 | 36 | 32 | — | 0 | — | — | 54 |
| D40/CT29 | — | 20 | — | 13 | — | 0 | — | — | — | 41 | — | 36 | 27 | — | — | — | 55 |
| HCA661/CT30 | 0 | — | — | — | — | 0 | 0 | 29 | — | — | 20 | — | — | — | — | — | 56 |
| PLU-1/CT31 | — | — | 86 | — | — | — | — | — | — | — | — | — | — | — | — | — | 27 |
| LDHC/CT32 | — | — | 35 | 15 | — | — | — | — | — | 47 | 44 | 42 | — | 37 | 57 | — | 18 |
| MORC/CT33 | — | — | 0 | 0 | — | — | — | — | — | 18 | 18 | 14 | — | 0 | 0 | — | 18 |
| SGY-1/CT34 | — | — | 20 | 0 | — | — | — | — | — | 12 | 25 | 57 | — | 12 | 0 | — | 18 |
| SPO11/CT35 | — | — | 0 | 0 | — | — | — | — | — | 0 | 6 | 0 | — | 0 | 0 | — | 18 |
| TPX1/CT36 | — | — | 15 | 0 | — | — | — | — | — | — | 6 | 14 | — | 37 | 14 | — | 18 |
| NYSAR35/CT37 | 42 | — | 23 | 0 | 8 | — | — | — | — | 17 | 6 | 8 | — | — | 0 | 8 | 57 |
| FTHL17/CT38 | 22 | — | 14 | 0 | 0 | — | 10 | — | 0 | 25 | 0 | — | — | 0 | 0 | 0 | 58 |
| NXF2/CT39 | 19 | — | 0 | 11 | 12 | — | 5 | — | 0 | 15 | 55 | — | — | 14 | 0 | 27 | 58 |
| TAF7L/CT40 | 10 | — | 0 | 0 | 0 | — | 10 | — | 0 | 9 | 21 | — | — | 0 | 0 | 12 | 58 |
| TDRD1/CT41.1 | 28 | — | 37 | 0 | 10 | — | 22 | — | 5 | 5 | 0 | — | — | 38 | 0 | 0 | 58 |
| TEX15/CT42 | 21 | — | 0 | 0 | 20 | — | 11 | — | 0 | 21 | 27 | — | — | 12 | 33 | 28 | 58 |
| FATE/CT43 | — | — | — | 21 | — | 7 | — | 66 | — | 0 | — | — | — | — | — | — | 19 |
| TPTE/CT44 | — | — | — | 0 | — | 0 | — | 39 | — | 36 | — | — | — | — | — | — | 19 |

^{a}Abbreviations: Blad, bladder; Brn, brain; Brst, breast; Col, colon; Gas, gastric; H/N, head and neck; Leuk, leukemia; Lymph, lymphoma, NSCLC, non-small cell lung carcinoma; Mel, melanoma; Ov, ovarian; Pancr, pancreatic; Pros, prostate; Sarc, sarcoma; Ref, reference.
^{b}Reference 59.

Additional antigens associated with tumor neovasculature are VEGFR2 (vascular endothelial growth factor receptor 2) described in U.S. Pat. No. 6,342,221, which is hereby incorporated by reference in its entirety; and Tie-2, an endothelium specific receptor tyrosine kinase which is described in WO9943801, which is hereby incorporated by reference in its entirety.

One of skill in the art will appreciate that any other antigen or protein associated with vascular cells can be a target for the immunogenic compositions, including those that are presently known and those yet to be identified.

### Compositions

Immunogenic compositions, including, for example, vaccines, can be prepared using whole antigen or an epitopic peptide. Peptide immunogens can be readily prepared using standard peptide synthesis means known in the art, for example. Immunogens can be prepared commercially by one of numerous companies that do chemical synthesis. An example such a company is American Peptides, Inc., where the distributor is CLINALFA AG (Laufelfingen, Switzerland). The antigens or immunogens can be prepared in accordance with GMP standards and purity can be assessed by analytical HPLC. The product can be characterized by amino-acid analysis and tested for sterility and the absence of pyrogens.

An antigen may be delivered to an animal's system either directly or indirectly. For example, a polypeptide may be delivered directly as the polypeptide, or it may be delivered indirectly, for example, using a DNA construct or vector, or a recombinant virus that codes for the desired antigen. Any vector driving expression in a professional antigen presenting cell can be suitable for this purpose. In indirect delivery, the antigen is expressed in the cell, then presented by the MHC Class I on the surface of the cell to stimulate a CTL response. Expression of a secreted form of the antigen can be useful to induce an antibody response recognizing antigens that are membrane proteins.

In a preferred embodiment, an encoded antigen can be delivered in the form of a naked plasmid expression vector. Particularly useful constructs are disclosed in U.S. patent application Ser. No. 09/561,572, entitled “EXPRESSION VECTORS ENCODING EPITOPES OF TARGET-ASSOCIATED ANTIGENS;” U.S. patent application Ser. No. 10/292,413 (Pub. No. 20030228634 A1), entitled “EXPRESSION VECTORS ENCODING EPITOPES OF TARGET-ASSOCIATED ANTIGENS AND METHODS FOR THEIR DESIGN;” U.S. patent application Ser. No. 10/225,568 (Pub No. 2003-0138808); PCT Application No. PCT/US2003/026231 (Pub. No. WO 2004/018666); U.S. Pat. No. 6,709,844, entitled AVOIDANCE OF UNDESIRABLE REPLICATION INTERMEDIATES IN PLASMID PROPAGATION, and in U.S. patent application Ser. No. 10/026,066 (Pub. No. 20030215425 A1), entitled EPITOPE SYNCHRONIZATION IN ANTIGEN PRESENTING CELLS, each of which is hereby incorporated by reference in its entirety. Additional methodology, compositions, peptides, and peptide analogues are disclosed in U.S. Provisional Application No. ______, (Attorney Docket MANNK.038PR), filed on the same date as the instant application, entitled “SSX-2 PEPTIDE ANALOGS;” which is incorporated herein by reference in its entirety. Further methodology, compositions, peptides, and peptide analogues are disclosed in U.S. Provisional Application No. ______ (Attorney Docket MANNK.039PR), filed on the same date as the instant application, entitled “NY-ESO PEPTIDE ANALOGS;” which is incorporated herein by reference in its entirety. The feasibility of and general procedures related to the use of naked DNA for immunization are described in U.S. Pat. No. 5,589,466, entitled “INDUCTION OF A PROTECTIVE IMMUNE RESPONSE IN A MAMMAL BY INJECTING A DNA SEQUENCE” and in U.S. Pat. No. 5,679,647, entitled “METHODS AND DEVICES FOR IMMUNIZING A HOST AGAINST TUMOR-ASSOCIATED ANTIGENS THROUGH ADMINISTRATIONS OF NAKED POLYNUCLEOTIDES WHICH ENCODE TUMOR-ASSOCIATED ANTIGENIC PEPTIDES,” each of which is hereby incorporated by reference in its entirety. The former teaches only intramuscular or intradermal injection while the latter teaches only administration to skin or mucosa.

In a preferred embodiment, the antigen can be administered directly to the lymphatic system. Intranodal administration for the generation of CTL is taught in U.S. patent application Ser. Nos. 09/380,534 and 09/776,232 (Pub. No. 20020007173 A1), and in PCT Application No. PCTUS98/14289 (Pub. No. WO9902183A2) each entitled “A METHOD OF INDUCING A CTL RESPONSE,” each of which is hereby incorporated by reference in its entirety. Single bolus injection intra lymph node (i.ln.) required only 0.1% of the dose required in order to obtain a similar level of CTL response by intramuscular (i.m.) injection. Therefore a protective response can be established against systemic viral infection with a single bolus delivered i.ln., but not with a dose nearing the practical limit delivered i.m. Repeated bolus injections i.m. failed to establish a protective response against a peripheral virus infection or transplanted tumor, whereas lower doses administered i.ln. were completely effective. Particularly useful intranodal immunization protocols are taught in Provisional U.S. Patent Application No. 60/479,393, entitled “METHODS TO CONTROL MAGNITUDE AND QUALITY THE MHC CLASS I-RESTRICTED IMMUNE RESPONSE,” and in U.S. patent application No. ______ entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE” (Attorney Docket No. MANNK.034A) (Pub. No. ______), filed on date even with the instant Application, each of which is hereby incorporated by reference in its entirety.

A class of epitopes that can be advantageous in anti-cancer immunogenic compositions are housekeeping epitopes. These are produced through the action of the housekeeping (or standard) proteasome. Housekeeping epitopes can be liberated from the translation product of expression vectors through proteolytic processing by the immunoproteasome of professional antigen presenting cells (pAPC). In one embodiment of the invention, sequences flanking the housekeeping epitope(s) can be altered to promote cleavage by the immunoproteasome at the desired location(s). Housekeeping epitopes, their uses, and identification are described in U.S. patent application Ser. No. 09/560,465 filed on Apr. 28, 2000, and U.S. patent application Ser. No. 10/026,066 (Pub. No. 20030215425 A1), filed on Dec. 7, 2001, entitled “EPITOPE SYNCHRONIZATION IN ANTIGEN PRESENTING CELLS,” and U.S. patent application Ser. No. 09/561,074, filed on Apr. 28, 2000, entitled “METHOD OF EPITOPE DISCOVERY,” each of which is hereby incorporated by reference in its entirety.

Examples of housekeeping epitopes are disclosed in Provisional U.S. Patent Applications Nos. 60/282,211, filed on Apr. 6, 2001; 60/337,017, filed on Nov. 7, 2001; 60/363,210 filed Mar. 7, 2002; and 60/409,123, filed on Sep. 5, 2002; U.S. patent application Ser. No. 10/117,937 (Publication No. 20030220239A1), filed on Apr. 4, 2002; and U.S. patent application Ser. No. 10/657,022, and PCT Application No. PCT/US2003/027706 (Pub. No. WO04022709A2) both entitled EPITOPE SEQUENCES; each of which is hereby incorporated by reference in its entirety.

In other embodiments of the invention, the housekeeping epitope(s) can be flanked by arbitrary sequences or by sequences incorporating residues known to be favored in immunoproteasome cleavage sites. As used herein the term “arbitrary sequences” refers to sequences chosen without reference to the native sequence context of the epitope, their ability to promote processing, or immunological function. In further embodiments of the invention multiple epitopes can be arrayed head-to-tail. These arrays can be made up entirely of housekeeping epitopes. Likewise, the arrays can include alternating housekeeping and immune epitopes. Alternatively, the arrays can include housekeeping epitopes flanked by immune epitopes, whether complete or distally truncated. Further, the arrays can be of any other similar arrangement. There is no restriction on placing a housekeeping epitope at the terminal positions of the array. The vectors can additionally contain authentic protein coding sequences or segments thereof containing epitope clusters as a source of immune epitopes. The term “authentic” refers to natural protein sequences.

Epitope clusters and their uses are described in U.S. patent application Ser. Nos. 09/561,571, entitled “EPITOPE CLUSTERS,” filed on Apr. 28, 2000; 10/005,905, entitled “EPITOPE SYNCHRONIZATION IN ANTIGEN PRESENTING CELLS,” filed on Nov. 7, 2001; and 10/026,066, filed on Dec. 7, 2001, also entitled “EPITOPE SYNCHRONIZATION IN ANTIGEN PRESENTING CELLS,” each of which is hereby incorporated by reference in its entirety.

In another embodiment of the invention an encoded antigen can be delivered in the form of a viral vector. A wide array of viruses with modified genomes adapted to express interposed reading frames but often no, or at least a reduced number of, viral proteins are known in the art, including without limitation, retroviruses including lentiviruses, adenoviruses, parvoviruses including adeno-associated virus, herpesviruses, and poxviruses including vaccinia virus. Such viral vectors facilitate delivery of the nucleic acid component into the cell allowing for expression. A subset of these vectors, such as retroviruses and parvoviruses, promote integration of their nucleic acid component into the host genome, whereas others do not.

Bacteria can also serve as vectors, that is they can be used to deliver a nucleic acid molecule capable of causing expression of an antigen. For example, a strain of Listeria monocytogenes has been devised that effects its own lysis upon entering the cytosol of macrophages (its normal target), thereby releasing plasmid from which antigen is subsequently expressed (Dietrich, G. et al., Biotechnology 16:181-185, 1998 which is hereby incorporated by reference in its entirety). Shigela flexneri and Escherichia coli have been similarly used (Sizemore, D. R. et al., Science 270:299-302, 1995, and Courvalin, P. et al., Life Sci. 318:1207-1212, 1995, respectively, each of which is hereby incorporated by reference in its entirety).

The use of microbial vectors for nucleic acid delivery can be complicated by the immune reactions the vectors themselves provoke. When prolonged or repeated administration is required, antibody elicited by the earlier treatment can prevent useful quantities of the vector from ever reaching its intended host. However, by direct administration into a lymph node, for example, the combination of proximity to host cells and the much reduced effective dose makes it possible to administer a dose capable of evading or overwhelming an existing antibody titer.

The word vector has been used, here and elsewhere, in reference to several modalities and variously modified (e.g., expression vector, viral vector, delivery vector, etc.). The underlying principle is that a nucleic acid capable of causing expression of an antigen, rather than the antigen itself, ultimately arrives in an APC. Unless modified, explicitly or by local context, the term vector as used herein is intended to encompass all such possibilities.

The techniques discussed above are distinct from the approach of modifying the microbial genome, including extra-chromosomal DNA, such that the antigen is produced as a component of the microbe, which is then itself administered as the immunogen. Examples of microbes used in the genomic modification approach include viruses, bacteria, fungi, and protazoa. In embodiments of the invention described herein, the compositions, including the vaccines, can include the already synthesized antigen or a nucleic acid capable of causing an APC to express the antigen in vivo. In alternative embodiments, combinations of these two techniques are used. For example, one embodiment contemplates the use of a virus vector as discussed above that also incorporates a target epitope into a capsid or envelope protein.

Antigens may be used alone or may be delivered in combination with other antigens or with other compounds such as cytokines. Cytokines that are known to enhance immune stimulation of CTL responses, include, for example, GM-CSF, IL-12, IL-2, TNF, IFN, IL-18, IL-3, IL-4, IL-8, IL-9, IL-13, IL-10, IL-14, IL-15, G-SCF, IFN alpha, IFN beta, IFN gamma, TGF alpha, TGF beta, and the like. Cytokines are known in the art and are readily available in the literature or commercially. Many animal and human tumors have been shown to produce cytokines, such as IL-4, IL-10, TGF-B, that are potent modulators of the immune response and that protect tumors from immune-mediated destruction. The production of IL-4, IL-10 or TGF-B by tumors may achieve this protective effect by suppressing the induction of cellular immunity, including the elaboration of CTL responses. Alternatively, cytokines that support CTL responses can be exogenously added to help in the balance between induction of anti-tumor cell mediated and non-tumor-destructive humoral responses. Several such exogenous cytokines show utility in experimental mouse vaccination models which are known to enhance CTL responses, including GM-CSF, IFN and IL-2. An example of an effective exogenous cytokine that may be used is GM-CSF. GM-CSF is reported to enhance the expression of the so called “co-stimulatory” molecules, such as B7-1 or B7-2 on antigen presenting cells (APC). These co-stimulatory molecules are important players in the variety of interactions that occur during stimulation of CTL by APC. Moreover, GM-CSF is known to induce activation of APCs and to facilitate growth and differentiation of APCs, thereby making these APCs important CTL stimulating cells available both in greater numbers and potency.

### Delivery of the Antigen

While not wanting to be bound by any particular theory, it is thought that T cells do not have a functional memory that is long-lived. Antibody-mediated B-cell memory, on the other hand, appears to have a long-lived effector memory. Thus, delivering an antigen that induces a CTL response is most preferably done over time to keep the patient's immune system appropriately stimulated to attack the target cells. In one approach the presence of antigen is maintained virtually continuously within the lymphatic system to maintain effector CTL function as disclosed in U.S. patent application Ser. No. 09/776,232 (Pub. No. 20020007173 A1), entitled “A METHOD OF INDUCING A CTL RESPONSE,” which is hereby expressly incorporated by reference. In another approach T cell memory is repeatedly induced, and re-amplified and reactivated as described in Provisional U.S. Patent Application No. 60/479,393, entitled “METHODS TO CONTROL MAGNITUDE AND QUALITY THE MHC CLASS I-RESTRICTED IMMUNE RESPONSE,” and in U.S. patent application No. ______ entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE” (Attorney Docket No. MANNK.034A) (Pub. No. ______), filed on date even with the instant Application, each of which is hereby incorporated by reference in its entirety. While it has been suggested that antigens and adjuvants can be prepared as biodegradable microspheres or liposomes, none of these preparations have thus far provided a CTL response that is useful for attacking cancer cells or pathogens on a long term basis. Preferably, delivery of the antigen is sustained over the desired period of time at a level sufficient to maintain the antigen level to obtain the desired response. In one embodiment, a reservoir having fluid antigen composition can be used to deliver the antigen such that it reaches the animal's lymphatic system. While much of the following discussion focuses on the use of infusion to deliver the antigen it is also possible to use bolus injections directly into the lymphatic system, the number and frequency of which will depend on the persistence of antigen conferred by the particular form and formulation of antigen used.

Ultimately antigen finds its way into the lymphatic system in order to most efficiently stimulate CTL. Delivery of antigen can involve infusion into various compartments of the body, including but not limited to subcutaneous, intravenous, intraperitoneal and intralymphatic, the latter being preferred. While each of these points of infusion results in antigen uptake into the lymphatic system, the relative amounts of antigen needed to induce a beneficial CTL response varies according to the site of infusion. In general, direct infusion of antigen into the lymph system is deemed to be the most efficient means of inducing a CTL response, however, any delivery route may be used. Pump systems are capable of delivering material quantities of antigen in a range that is suitable for inducing a CTL response through delivery to all compartments of the body. CTL stimulation following delivery of antigen via the various routes will vary depending on the properties of different antigens, including factors that influence antigen behavior in the body and its rate of equilibration to (or longevity in) the lymph, such as antigen stability in the body fluid, solubility of antigen in body fluid, binding affinity for HLA and potency as a stimulator of CTL.

In a preferred embodiment, introduction of the antigen is done as directly as possible to the lymphatic system to avoid the destruction of the antigen by metabolism in the body. When introduction of a fluid antigen composition occurs subcutaneously, larger quantities of antigen are needed to assure enough antigen reaches the lymphatic system. Such subcutaneous injection is contemplated by the invention disclosed herein, depending on factors such as cost, stability of the antigen, how quickly the antigen gets to the lymph system, how well it equilibrates with the lymph, and other factors that the attending doctor or specialist will recognize. Subcutaneous delivery generally can require 100 to 1000 times more antigen than direct delivery to the lymph system. It is preferable, therefore, that the antigen composition is introduced through a device for local administration to the lymphatic system, e.g., the spleen, a lymph node, or a lymph vessel. The device for local administration can be positioned outside the patient or implanted into the patient. In either case, the device can have a reservoir to hold the fluid antigen-containing composition, a pump to transfer the composition, and a transmission channel leading from the reservoir to be directed to the preferred region of administration in the patient's body. In either case it is preferably portable.

For the device positioned outside the patient's body (the external device), there are numerous devices used for delivering insulin to diabetic patients that are useful in delivering antigen according to the embodiments described herein. Generally these devices can be comprised of a reservoir for holding the antigen composition (instead of insulin), a programmable pump to pump the composition out of the reservoir, a transmission channel or line for transmitting the composition, and a means to introduce the composition into the animal's body to ultimately reach the lymphatic system.

Preferably, the reservoir for the antigen composition should be large enough for delivery of the desired amount of antigen over time and easily refillable or replaceable without requiring the user to reinsert the means for introducing the antigen composition to the lymph system.

In preparing the antigen compositions of embodiments of the invention disclosed herein, a composition (preferably aqueous) can be prepared to be compatible with the lymph system and physiologically acceptable to the animal being treated. Relevant considerations include, for example, the physicochemical properties of the antigen, such as the isoelectric point, molecular weight, glycosylation or other post-translational modification, and overall amino acid composition. These properties along with any known behavior of the drug in different solutions (e.g., different buffers, cofactors, etc.) as well as its in vivo behavior can help guide the choice of formulation components. One parameter that impacts all the major degradation pathways is the solution pH. Thus, the initial formulations also assess the pH dependence of the degradation reactions and the mechanism for degradation, which can often be determined from the pH dependence to determine the stability of the protein in each solution. Rapid screening methods usually involve the use of accelerated stability at elevated temperatures (e.g., 40° C.) using techniques known in the art.

In general the antigen compositions useful in embodiments described herein can be suitable for parenteral injection, in very small quantities. As such a composition should be free of contamination and have a pH compatible with the lymphatic system. However, because very small quantities of the antigenic composition will be delivered it need not be the same pH as blood or lymph, and it need not be aqueous-based. The preferable pH range that is compatible is from about 6.7-7.3 and can be prepared using water for injection to meet USP specifications (see Remington: The Science and Practice of Pharmacy, Nineteenth Edition; Chapters 86-88). For antigens that are less soluble, a suitable cosolvent or surfactant may be used, such as dimethyl sulfoxide (DMSO) or PLURONIC brand surfactants. Generally, a standard saline solution that is buffered with a physiologically acceptable weak acid and its base conjugate, e.g., a phosphate or citrate buffering system, will be the basis of the antigen composition. In some cases, a small amount of an antioxidant may be useful to stabilize the composition and prevent oxidation. Factors to consider in preparing the antigen compositions may be found in the 1994 American Chemical Society book entitled “Formulation and Delivery of Proteins and Peptides” (Acs Symposium Series, No. 567) by Jeffery L. Cleland and Robert Langer (Editor)).

For nucleic acid encoded antigens similar considerations can apply, although the variety of physico-chemical properties encountered with polypeptides is absent, so that acceptable formulations will have nearly universal applicability. As seen in Examples 6-10, plasmid DNA in standard phosphate buffered saline (PBS) is an acceptable and effective formulation. In some embodiments of the invention, DNA is administered continuously or intermittently at short intervals, from a reservoir worn on, or implanted in, the patient's body. It is preferable that the DNA be maintained in a soluble, stable form at or near body temperature over a period of time measured minimally in days. In such applications where the formulated nucleic acid will be delivered from a reservoir over a period of several days or longer, the stability of the nucleic acid at room or body temperature for that period of time, as well as its continued sterility, take on increased importance. The addition of bacteriostatic agents (e.g., benzyl or ethyl alcohol) and chelating agents (e.g. EDTA) is useful toward these ends. Formulations containing about 0.5-2% ethyl alcohol, 0.25-0.5 mM EDTA generally perform well. Such formulations are also appropriate for bolus injections.

Generally the amount of the antigen in the antigen composition will vary from patient to patient and from antigen to antigen, depending on such factors as the activity of the antigen in inducing a response and the flow rate of the lymph through the patient's system. In general the antigen composition may be delivered at a rate of from about 1 to about 500 microliters/hour or about 24 to about 12000 microliters/day. The concentration of the antigen is such that about 0.1 micrograms to about 10,000 micrograms of the antigen will be delivered during 24 hours. The flow rate is based on the knowledge that each minute approximately about 100 to about 1000 microliters of lymph fluid flows through an adult inguinal lymph node. The objective is to maximize local concentration of vaccine formulation in the lymph system. A certain amount of empirical investigation on patients will be necessary to determine the most efficacious level of infusion for a given vaccine preparation in humans.

To introduce the antigen composition into the lymphatic system of the patient the composition is preferably directed to a lymph vessel, lymph node, the spleen, or other appropriate portion of the lymph system. Preferably, the composition is directed to a lymph node such as an inguinal or axillary node by inserting a catheter or needle to the node and maintaining the catheter or needle throughout the delivery. Suitable needles or catheters are available made of metal or plastic (e.g., polyurethane, polyvinyl chloride [PVC], TEFLON, polyethylene, and the like). In inserting the catheter or needle into the inguinal node for example, the inguinal node is punctured under ultrasonographic control using a Vialon™ Insyte-W™ cannula and catheter of 24G3/4 (Becton Dickinson, USA) which is fixed using Tegaderm™ transparent dressing (Tegaderm™ 1624, 3M, St. Paul, Minn. 55144, USA). This procedure is generally done by an experienced radiologist. The location of the catheter tip inside the inguinal lymph node is confirmed by injection of a minimal volume of saline, which immediately and visibly increases the size of the lymph node. The latter procedure allows confirmation that the tip is inside the node. This procedure can be performed to ensure that the tip does not slip out of the lymph node and can be repeated on various days after implantation of the catheter. In the event that the tip does slip out of location inside the lymph node, a new catheter can be implanted.

### Formulation and Treatment Protocol

There are several approaches to utilizing the combination of TuAAs with DNA vaccines. A first approach is to include all the antigens or epitopes from all the antigens in a given combination into a single DNA expression vector. This approach has the advantages of simplicity for manufacturing and administration to patients. However, in some instances, epitope competition can limit the usefulness of this approach. That is, it is possible that only the most immunogenic epitope will elicit an immune response when a vaccine with several epitopes representing all TuAAs in the combination is given to patients. It is also more difficult to design and construct a DNA vaccine in which all epitopes are expressed at high efficiencies. Nevertheless, because the procedure for treating patients is simple and uniform within each type of cancer, the cost is likely to be lower than for the other approaches described below.

An alternate approach is to include only one antigen or epitopes of one antigen in a DNA expression vector. This approach has the advantages of simplicity in designing and constructing the DNA vector, flexibility, and customized administration to patients. If a large number of individual TuAA vaccines are available, then one can customize treatment for each individual patient based on the TuAA expression profile of his or her tumor. For example, if the standard combination for treating a given type of cancer is TuAA A, B, and C (where A, B, and C designate different tumor associated antigens), but a patient's tumor expresses TuAA A, C, and Z (but not B), then the patient can be treated with separate vaccines for each of A, C, and Z. It is expected that this flexibility and customizability will improve the success rate of immunotherapy because antigen redundancy can be achieved for each patient. However, the procedure of treating the patient may be more complex. For example, delivery using this approach may include a sequential administration scheme (one antigen at a time), or injection into multiple, anatomically separate sites of the patient at about the same time.

Still another approach is to combine epitopes from multiple TuAAs that have similar immunogenicity into a DNA expression vector (more than one vector may be used for some combinations). This approach can have some of the advantages of the above two approaches but also can suffer from the disadvantages of the previous two.

A profile of the antigen expression of a particular tumor can be used to determine which antigen or combination of antigens to use. Exemplary methodology is found in U.S. Provisional Application No. ______ (Attorney Docket: MANNK.035PR2), filed on even date herewith, entitled “COMBINATIONS OF TUMOR-ASSOCIATED ANTIGENS IN DIAGNOSTICS FOR VARIOUS TYPES OF CANCERS;” and which is hereby incorporated by reference in its entirety.

Patients that can benefit from such methods of immunization can be recruited using methods to define their MHC protein expression profile and general level of immune responsiveness. In addition, their level of immunity can be monitored using standard techniques in conjunction with access to peripheral blood. Finally, treatment protocols can be adjusted based on the responsiveness to induction or amplification phases and variation in antigen expression. For example, repeated entrainment doses preferably can be administered until a detectable response is obtained, and then administering the amplifying peptide dose(s), rather than amplifying after some set number of entrainment doses. Similarly, scheduled amplifying or maintenance doses of peptide can be discontinued if their effectiveness wanes, antigen-specific regulatory T cell numbers rise, or some other evidence of tolerization is observed, and further entrainment can be administered before resuming amplification with the peptide. The integration of diagnostic techniques to assess and monitor immune responsiveness with methods of immunization is discussed more fully in Provisional U.S. patent application No. ______ (Atty. Docket No. MANNK.040PR), entitled IMPROVED EFFICACY OF ACTIVE IMMUNOTHERAPY BY INTEGRATING DIAGNOSTIC WITH THERAPEUTIC METHODS, which was filed on date even with the present application and is hereby incorporated by reference in its entirety.

Many variations and alternative elements of the invention have been disclosed. Still further variations and alternate elements will be apparent to one of skill in the art. Various embodiments of the invention can specifically include or exclude any of these variation or elements.

Each of the references cited herein is hereby incorporated herein by reference in its entirety.

The following examples are for illustrative purposes only and are not intended to limit the scope of the embodiments in any way.

### EXAMPLES

### TuAA Analysis and Selection of Combinations

The presence of TuAAs was measured by Real-Time PCR(RT-PCR). Briefly, total RNA was isolated from tumor specimens by standard methods and cDNA was made with standard reverse transcription procedures. Complementary DNA (cDNA) was amplified with specially designed, gene specific, primers that anneal only to cDNA but not genomic DNA. TuAA expression patterns of 12 ovarian and 7 colorectal tumor specimens were analyzed by RT-PCR. The results are summarized in the Table 4 below.

TABLE 4

| | Total # | PRAME | NY-ESO-1 | SSX-2 | PSMA | MAGE1 | MAGE3 |
|---|---|---|---|---|---|---|---|
| Ovarian | 12 | 12 | 5 | 6 | 6 | 4 | 3 |
| Colorectal | 7 | 5 | 1 | 2 | 5 | 0 | 1 |

### Example 1

### Ovarian Cancer

In the case of ovarian cancer, all samples analyzed were positive for PRAME. Thus the inclusion of PRAME in the combination improves coverage of the cases with ovarian cancer.

In order to achieve antigen redundancy and improve coverage in a large population, combinations of other antigens were considered in addition to PRAME. SSX-2 as well as PSMA were present in 6 of the 12 cases individually, but the combination of SSX-2 and PSMA provided coverage in 9 of 12 cases. Although NY-ESO-1 and SSX-2 were only present in 5 and 6 of the 12 cases, respectively, either NY-ESO-1 or SSX-2 was detected in 7 of the 12 cases.

Thus, the combination of PRAME, SSX-2, and PSMA or PRAME, NY-ESO-1, and SSX-2 provided preferable coverage and redundancy compared to the combination of PRAME and PSMA or the combination of PRAME and SSX-2. The combination of PRAME, SSX-2, and PSMA provided excellent coverage of cases and good antigen redundancy because the majority of ovarian tumor samples analyzed had at least two of the four TuAA in the combination present. The combination of PRAME, SSX-2, PSMA, and NY-ESO-1 provided more preferred antigen redundancy, and thus, lower possibility of tumor escape.

### Example 2

### Colorectal Cancer

In the case of colorectal cancer, PRAME and PSMA were each detected in 5 of the 7 samples analyzed. In 6 of the 7 cases, either PRAME or PSMA was detected. Although SSX-2 was only detected in 2 of 7 cases, both SSX-2-PRAME and SSX2-PSMA combinations increased coverage to 6 of 7. Similarly, although NYESO-1 was detected in only 1 of 7 cases, the combination of NY-ESO-1-PRAME as well as the NYESO-1-PSMA combination increased coverage to 6 of 7. The addition of SSX-2 or NYESO-1 to the PRAME and PSMA combination improved coverage to 7 of 7. Thus, the combination of PRAME, PSMA, and NYESO-1, or the combination of PRAME, PSMA, and SSX-2 provided good coverage of cases and redundancy of antigens for a majority of patients. The combination of PRAME, PSMA, NY-ESO-1, and SSX-2 provided further redundancy.

### Example 3

### Pancreatic Cancer

In pancreatic cancer specimens, the presence of NYESO-1 and SSX-2 was detected in 40% and 20% of the specimens, respectively. PSMA and over-expression of HER2-/neu were reported to be present in 100% and 21% of pancreatic tumors, respectively (Chang S S et al, Cancer Res 1999, 59:3192; Safran H et al, Am J Clin Oncol. 2001, 24:496). Although over-expression of HER2/neu may render the cancer tissue a preferred target, thus providing some specificity for immunotherapy, low level expression of HER2/neu in normal tissues remains a concern. Thus, the combination of NYESO-1, SSX-2, and PSMA provides excellent coverage and some redundancy for treating pancreatic cancer.

### Example 4

### Non-Small Cell Lung Cancer

For non-small cell lung cancer, the reported presence of NYESO-1, SSX-2, MAGE-3, BAGE, over-expression of Her2/neu, and PSMA was 21, 15, 60, 6, 16, and 100%, respectively (Scanlan M J et al, Cancer lett 2000, 150:155; Chang S S et al, Cancer Res 1999, 59:3192; Selvaggi G et al, Cancer 2002, 94:2669, which are hereby incorporated by reference in their entireties). Thus, the combination of NYESO-1, SSX-2, MAGE-3, and PSMA provides coverage and antigen redundancy for the immunotherapy of non-small cell lung cancer.

### Example 5

### Renal Cell Carcinoma

For renal cell carcinoma, SSX-2, PSMA and PRAME were detected with frequencies of 5, 100 and 40%, respectively (Sahin, U et al, Clin Cancer Res. 2000, 6:3916; Chang S S et al, Urology 2001, 57:801; Neumann E et al, Cancer Res. 1998, 58:4090, which are hereby incorporated by reference in their entireties). Thus, the combination of PSMA and PRAME provides excellent coverage and redundancy for renal cell carcinoma. Adding SSX-2 to the combination of PSMA and PRAME improves redundancy.

### Example 6

### Melanoma

For melanoma, Melan A, Tyrosinase, NYESO-1, and SSX-2 were reported to be present in 92, 92, 41, and 35% of tumor specimens, respectively (Fetsch P A, et al, Cancer 1999, 87:37; Fetsch P A, et al, Cancer 2000, 90:252; Schultz-Thater E et al, Br J Cancer 2000, 83:204; Sahin, U et al, Clin Cancer Res. 2000, 6:3916). Therefore, the combination of Melan A, Tyrosinase, NYESO-1, and SSX-2 provides excellent coverage and antigen redundancy for the immunotherapy of melanoma. Significant redundancy is achieved using tyrosinase and melan-A together, or by combining NY-ESO-1 and SSX-2 with either of tyrosinase or melan-A.

### Example 7

### Schedule of Immunization with Plasmids Expressing Epitopes from Two Antigens

Two groups of HHD mice (n=4) were immunized via intra lymph node injection with either pSEM expressing Melan-A₂₆₋₃₅A27L (ELA) and pCBP expressing SSX-2₄₁₋₄₉ as a mixture; or with pSEM in the left inguinal lymph node and pCBP in the right inguinal lymph node, twice, at day 0 and 4 as shown in FIG. 1. The amount of the plasmid was 25 μg/plasmid/dose. Two weeks later, the animals were sacrificed, and cytotoxicity was measured against T2 cells pulsed or not with peptide.

### Example 8

### Co-Administration of Different Vectors Carrying Distinct Antigens

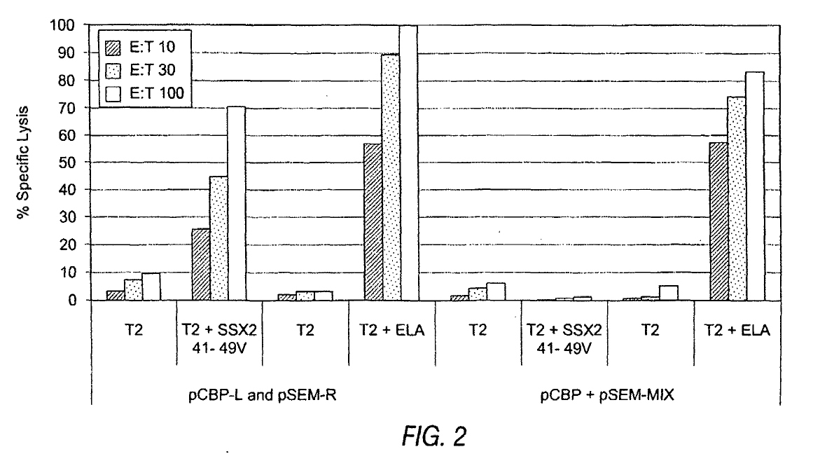

The animals immunized as described in Example 7, were sacrificed and splenocytes from each group pooled and stimulated with the two peptides (ELA or SSX-2₄₁₋₄₉) in parallel. The cytotoxicity was measured by incubation with Cr⁵¹-tagged, peptide loaded T2 target cells. Data in FIG. 2 show mean of specific cytotoxicity (n=4/group) against various target cells.

The results show that use of plasmid mixture interferes with the response elicited by pCBP plasmid; however, segregating the two plasmids relative to site of administration rescues the activity of pCBP. Thus, the co-administration of different vectors carrying distinct antigens can result in establishment of a hierarchy with regard to immunogenicity. Vector segregation can rescue the immunogenicity of the less dominant component, resulting in a multivalent response.

### Example 9

### Rescue of Multivalent Response by Addition of Peptide Boost Steps

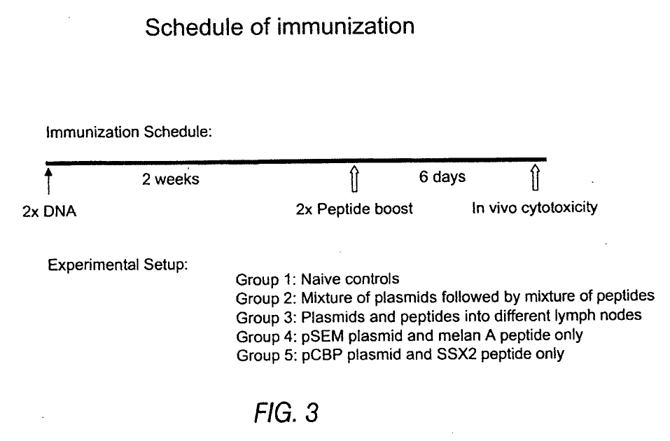

Four groups of HHD mice (n=6) were immunized via intra lymph node injection with either pSEM and pCBP as a mixture; or with pSEM in the left inguinal lymph node and pCBP in the right inguinal lymph node, twice, at day 0 and 4 as shown in FIG. 3. As a control, mice were immunized with either pSEM or pCBP plasmid. The amount of the plasmid was 25 μg/plasmid/dose. Two weeks later, the animals were boosted with melan A and/or SSX-2 peptides, mirroring the plasmid immunization dose and combination. Two weeks later, the animals were challenged with splenocytes stained with CFSE and loaded or not with Melan A or SSX-2 peptide, for evaluation of in vivo cytotoxicity.

### Example 10

### Peptide Amplification Rescues the Immunogenicity of the Less Dominant Epitope

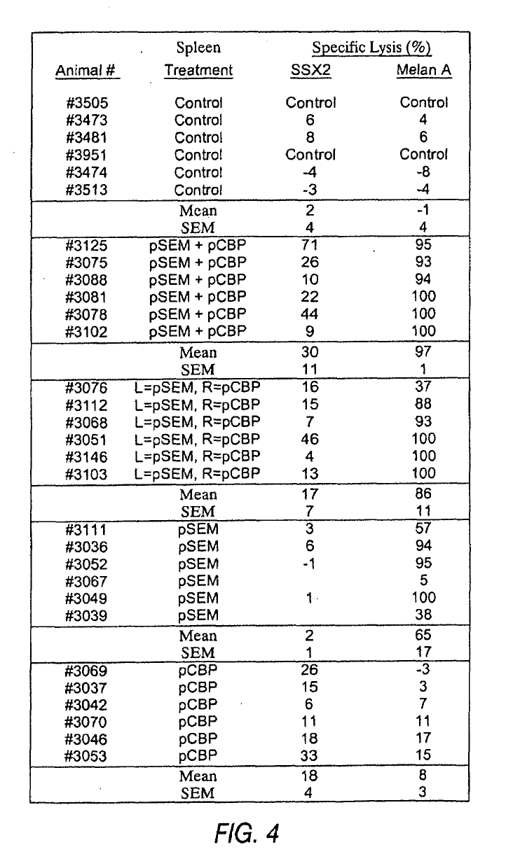

Mice were immunized as described in Example 9 and challenged with HHD littermate splenocytes coated with ELA or SSX-2 peptide, employing a triple peak CFSE in vivo cytotoxicity assay that allows the assessment of the specific lysis of two antigen targets simultaneously. Equal numbers of control-CFSE^{lo}, SSX-2₄₁₋₄₉-CFSE^{med}, and ELA-CFSE^{hi} cells were intravenously infused into immunized mice and 18 hours later the mice were sacrificed and target cell elimination was measured in the spleen (FIG. 4) by CFSE fluorescence using a flow cytometry. FIG. 4 shows the percent specific lysis of the SSX2 and Melan-A antigen targets from individual mice, as well as the mean and SEM for each group.

The results show that immunizing the animals with a mixture of the two vaccines comprising plasmids followed by peptides generated immunity to both antigens and resulted in the highest immune response, representing an average SSX-2 percent specific lysis in the spleen of 30+/−11, and an average Melan-A percent specific lysis of 97+/−1.

### Example 11

### Clinical Practice for Entrain-and-Amplify Immunization

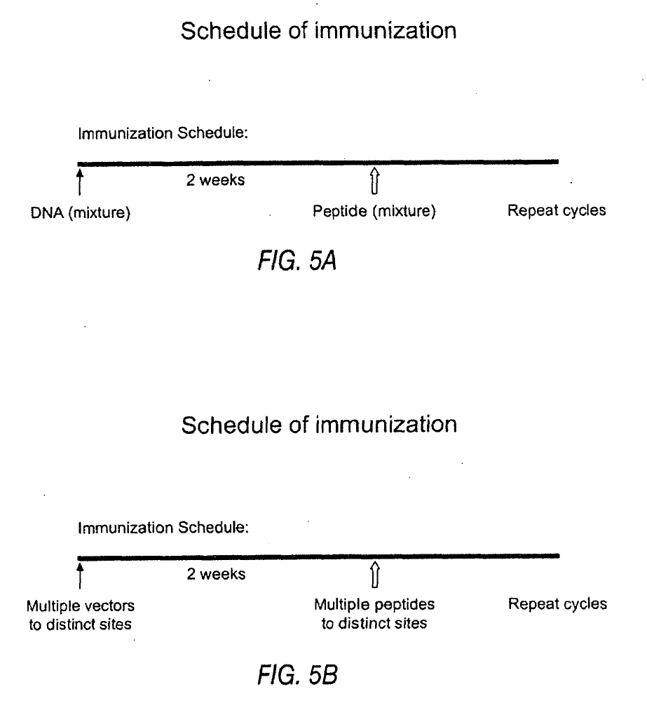

The data in FIGS. 2 and 4 suggest two scenarios for achieving a strong multivalent response in the clinic, shown in FIG. 5. In the first scenario (A), use of peptides for boosting restores multivalent immune responses even if plasmids and peptides are used as mixtures. In the second scenario (B), segregation of plasmid and peptide components respectively, allows induction of multivalent immune responses.

### Example 12

### Antigen Sequences

Below are sequences of the antigens listed in Table 1.

SEQ. ID NO. 1
SIZE: 529 aa
DEFINITION Tyrosinase precursor
ACCESSION NO.: P14679
mllavlycll wsfqtsaghf pracvssknl mekeccppws
gdrspcgqls grgscqnill snaplgpqfp ftgvddresw
psvfynrtcq csgnfmgfnc gnckfgfwgp ncterrllvr
rnifdlsape kdkffayltl akhtissdyv ipigtygqmk
ngstpmfndi niydlfvwmh yyvsmdallg gseiwrdidf
aheapaflpw hrlfllrweq eiqkltgden ftipywdwrd
aekcdictde ymggqhptnp nllspasffs swqivcsrle
eynshqslcn gtpegplrrn pgnhdksrtp rlpssadvef
clsltqyesg smdkaanfsf rntlegfasp ltgiadasqs
smhnalhiym ngtmsqvqgs andpifllhh afvdsifeqw
lrrhrplqev ypeanapigh nresymvpfi plyrngdffi
sskdlgydys ylqdsdpdsf qdyiksyleq asriwswllg
aamvgavlta llaglvsllc rhkrkqlpee kqpllmeked
yhslyqshl
SEQ. ID NO. 2
SIZE: 118 aa
DEFINITION Melanoma antigen recognized by T-cells
1 (MART-1) (Melan-A protein)
ACCESSION NO.: Q16655
mpredahfiy gypkkghghs yttaeeaagi giltvilgvl
lligcwycrr rngyralmdk slhvgtqcal trrcpqegfd
hrdskvslqe kncepvvpna ppayeklsae qspppysp
SEQ. ID NO. 3
SIZE: 223 aa
DEFINITION: synovial sarcoma, X breakpoint 2
isoform a; sarcoma, synovial, X-chromosome-related
2; 55X2 protein [Homo sapiens].
ACCESSION NO.: NP_003138
mngddafarr ptvgaqipek iqkafddiak yfskeewekm
kasekifyvy mkrkyeamtk lgfkatlppf mcnkraedfq
gndldndpnr gnqverpqmt fgrlqgispk impkkpaeeg
ndseevpeas gpqndgkelc ppgkpttsek ihersgnrea
qekeerrgta hrwssqnthn igrfslstsm gavhgtpkti
thnrdpkggn mpgptdcvre nsw
SEQ. ID NO. 4
SIZE: 750 aa
DEFINITION folate hydrolase (prostate-specific
membrane antigen) [Homo sapiens].
ACCESSION NO.: NP_004467
mwnllhetds avatarrprw lcagalvlag gffllgflfg
wfikssneat nitpkhnmka fldelkaeni kkflynftqi
phlagteqnf qlakqiqsqw kefgldsvel ahydvllsyp
nkthpnyisi inedgneifn tslfeppppg yenvsdivpp
fsafspqgmp egdlvyvnya rtedffkler dmkincsgki
viarygkvfr gnkvknaqla gakgvilysd padyfapgvk
sypdgwnlpg ggvqrgniln lngagdpltp gypaneyayr
rgiaeavglp sipvhpigyy daqkllekmg gsappdsswr
gslkvpynvg pgftgnfstq kvkmhihstn evtriynvig
tlrgavepdr yvilgghrds wvfggidpqs gaavvheivr
sfgtlkkegw rprrtilfas wdaeefgllg stewaeensr
llqergvayi nadssiegny tlrvdctplm yslvhnltke
lkspdegfeg kslyeswtkk spspefsgmp risklgsgnd
fevffqrlgi asgrarytkn wetnkfsgyp lyhsvyetye
lvekfydpmf kyhltvaqvr ggmvfelans ivlpfdcrdy
avvlrkyadk iysismkhpq emktysvsfd slfsavknft
eiaskfserl qdfdksnpiv lrmmndqlmf lerafidplg
lpdrpfyrhv iyapsshnky agesfpgiyd alfdieskvd
pskawgevkr qiyvaaftvq aaaetlseva
SEQ. ID NO. 5
SIZE: 309 aa
DEFINITION Melanoma-associated antigen 1 (MAGE-1
antigen) (Antigen MZ2-E).
ACCESSION NO.: P43355
msleqrslhc kpeealeaqq ealglvcvqa atssssplvl
gtleevptag stdppqspqg asafpttinf trqrqpsegs
ssreeegpst scileslfra vitkkvadlv gflllkyrar
epvtkaemle sviknykhcf peifgkases lqlvfgidvk
eadptghsyv lvtclglsyd gllgdnqimp ktgfliivlv
miamegghap eeeiweelsv mevydgrehs aygeprkllt
qdlvqekyle yrqvpdsdpa ryeflwgpra laetsyvkvl
eyvikvsarv rfffpslrea alreeeegv
SEQ. ID NO. 6
SIZE: 314 aa
DEFINITION Melanoma-associated antigen 3 (MAGE-3
antigen) (Antigen MZ2D)
ACCESSION NO.: P43357
mpleqrsqhc kpeeglearg ealglvgaqa pateeqeaas
ssstlvevtl gevpaaespd ppqspqgass lpttmnyplw
sqsyedssnq eeegpstfpd lesefqaals rkvaelvhfl
llkyrarepv tkaemlgsvv gnwqyffpvi fskassslql
vfgielmevd pighlyifat clglsydgll gdnqimpkag
lliiviaiia regdcapeek iweelsvlev fegredsilg
dpkklltqhf vqenyleyrq vpgsdpacye flwgpralve
tsyvkvlhhm vkisggphis ypplhewvlr egee
SEQ. ID NO. 7
SIZE: 180 aa
DEFINITION Cancer/testis antigen 1
(Autoimmunogenic cancer/testis antigen NYESO-1).
ACCESSION NO.: P78358
mqaegrgtgg stgdadgpgg pgipdgpggn aggpgeagat
ggrgprgaga arasgpggga prgphggaas glngccrcga
rgpesrllef ylampfatpm eaelarrsla qdapplpvpg
vllkeftvsg niltirltaa dhrqlqlsis sclqqlsllm
witqcflpvf laqppsgqrr
SEQ. ID NO. 8
SIZE: 509 aa
DEFINITION preferentially expressed antigen in
melanoma; melanoma antigen preferentially
expressed in tumors; Opa-interacting protein OIP4;
preferentially expressed antigen of melanoma
[Homo sapiens].
ACCESSION NO.: NP_006106
merrrlwgsi qsryismsvw tsprrlvela gqsllkdeal
aiaalellpr elfpplfmaa fdgrhsqtlk amvqawpftc
lplgvlmkgq hlhletfkav ldgldvllaq evrprrwklq
vldlrknshq dfwtvwsgnr aslysfpepe aaqpmtkkrk
vdglsteaeq pfipvevlvd lflkegacde lfsyliekvk
rkknvlrlcc kklkifampm qdikmilkmv qldsiedlev
tctwklptla kfspylgqmi nlrrlllshi hassyispek
eeqyiaqfts gqlslqclqa lyvdslfflr grldqllrhv
mnpletlsit ncrlsegdvm hlsqspsvsq lsvlslsgvm
ltdvspeplq allerasatl qdlvfdecgi tddqllallp
slshcsqltt lsfygnsisi salqsllqhl iglsnlthvl
ypvplesyed ihgtlhlerl aylharlrel lcelgrpsmv
wlsanpcphc gdrtfydpep ilcpcfmpn
SEQ. ID NO. 9
SIZE: 1255 aa3
DEFINITION Receptor protein-tyrosine kinase erbB-2
precursor (p185erbB2) (NEU proto-oncogene)
(C-erbB-2) (Tyrosine kinase-type cell surface
receptor HER2) (MLN 19).
ACCESSION NO.: P04626
melaalcrwg lllallppga astqvctgtd mklrlpaspe
thldmlrhly qgcqvvqgnl eltylptnas lsflqdiqev
qgyvliahng vrqvplqrlr ivrgtqlfed nyalavldng
dplnnttpvt gaspgglrel qlrslteilk ggvliqrnpq
lcyqdtilwk difhknnqla ltlidtnrsr achpcspmck
gsrcwgesse dcqsltrtvc aggcarckgp lptdccheqc
aagctgpkhs dclaclhfnh sgicelhcpa lvtyntdtfe
smpnpegryt fgascvtacp ynylstdvgs ctlvcplhnq
evtaedgtqr cekcskpcar vcyglgmehl revravtsan
iqefagckki fgslaflpes fdgdpasnta plqpeqlqvf
etleeitgyl yisawpdslp dlsvfqnlqv irgrilhnga
ysltlqglgi swlglrslre lgsglalihh nthlcfvhtv
pwdqlfrnph qallhtanrp edecvgegla chqlcarghc
wgpgptqcvn csqflrgqec veecrvlqgl preyvnarhc
lpchpecqpq ngsvtcfgpe adqcvacahy kdppfcvarc
psgvkpdlsy mpiwkfpdee gacqpcpinc thscvdlddk
gcpaeqrasp ltsiisavvg illvvvlgvv fgilikrrqq
kirkytmrrl lqetelvepl tpsgampnqa qmrilketel
rkvkvlgsga fgtvykgiwi pdgenvkipv aikvlrents
pkankeilde ayvmagvgsp yvsrllgicl tstvqlvtql
mpygclldhv renrgrlgsq dllnwcmqia kgmsyledvr
lvhrdlaarn vlvkspnhvk itdfglarll dideteyhad
ggkvpikwma lesilrrrft hqsdvwsygv tvwelmtfga
kpydgipare ipdllekger lpqppictid vymimvkcwm
idsecrprfr elvsefsrma rdpqrfvviq nedlgpaspl
dstfyrslle dddmgdlvda eeylvpqqgf fcpdpapgag
gmvhhrhrss strsgggdlt lglepseeea prsplapseg
agsdvfdgdl gmgaakglqs lpthdpsplq rysedptvpl
psetdgyvap ltcspqpeyv nqpdvrpqpp spregplpaa
rpagatlerp ktlspgkngv vkdvfafgga venpeyltpq
ggaapqphpp pafspafdnl yywdqdpper gappstfkgt
ptaenpeylg ldvpv
SEQ. ID NO. 10
SIZE: 2384 bp
DEFINITION Homo sapiens tyrosinase (oculocutaneous
albinism IA) (TYR), mRNA.
ACCESSION NO.: NM_000372
tattgagttct tcaaacatt gtagcctctt tatggtctct
gagaaataac taccttaaac ccataatctt taatacttcc
taaactttct taataagaga agctctattc ctgacactac
ctctcatttg caaggtcaaa tcatcattag ttttgtagtc
tattaactgg gtttgcttag gtcaggcatt attattacta
accttattgt taatattcta accataagaa ttaaactatt
aatggtgaat agagtttttc actttaacat aggcctatcc
cactggtggg atacgagcca attcgaaaga aaagtcagtc
atgtgctttt cagaggatga aagcttaaga taaagactaa
aagtgtttga tgctggaggt gggagtggta ttatataggt
ctcagccaag acatgtgata atcactgtag tagtagctgg
aaagagaaat ctgtgactcc aattagccag ttcctgcaga
ccttgtgagg actagaggaa gaatgctcct ggctgttttg
tactgcctgc tgtggagttt ccagacctcc gctggccatt
tccctagagc ctgtgtctcc tctaagaacc tgatggagaa
ggaatgctgt ccaccgtgga gcggggacag gagtccctgt
ggccagcttt caggcagagg ttcctgtcag aatatccttc
tgtccaatgc accacttggg cctcaatttc ccttcacagg
ggtggatgac cgggagtcgt ggccttccgt cttttataat
aggacctgcc agtgctctgg caacttcatg ggattcaact
gtggaaactg caagtttggc ttttggggac caaactgcac
agagagacga ctcttggtga gaagaaacat cttcgatttg
agtgccccag agaaggacaa attttttgcc tacctcactt
tagcaaagca taccatcagc tcagactatg tcatccccat
agggacctat ggccaaatga aaaatggatc aacacccatg
tttaacgaca tcaatattta tgacctcttt gtctggatgc
attattatgt gtcaatggat gcactgcttg ggggatctga
aatctggaga gacattgatt ttgcccatga agcaccagct
tttctgcctt ggcatagact cttcttgttg cggtgggaac
aagaaatcca gaagctgaca ggagatgaaa acttcactat
tccatattgg gactggcggg atgcagaaaa gtgtgacatt
tgcacagatg agtacatggg aggtcagcac cccacaaatc
ctaacttact cagcccagca tcattcttct cctcttggca
gattgtctgt agccgattgg aggagtacaa cagccatcag
tctttatgca atggaacgcc cgagggacct ttacggcgta
atcctggaaa ccatgacaaa tccagaaccc caaggctccc
ctcttcagct gatgtagaat tttgcctgag tttgacccaa
tatgaatctg gttccatgga taaagctgcc aatttcagct
ttagaaatac actggaagga tttgctagtc cacttactgg
gatagcggat gcctctcaaa gcagcatgca caatgccttg
cacatctata tgaatggaac aatgtcccag gtacagggat
ctgccaacga tcctatcttc cttcttcacc atgcatttgt
tgacagtatt tttgagcagt ggctccgaag gcaccgtcct
cttcaagaag tttatccaga agccaatgca cccattggac
ataaccggga atcctacatg gttcctttta taccactgta
cagaaatggt gatttcttta tttcatccaa agatctgggc
tatgactata gctatctaca agattcagac ccagactctt
ttcaagacta cattaagtcc tatttggaac aagcgagtcg
gatctggtca tggctccttg gggcggcgat ggtaggggcc
gtcctcactg ccctgctggc agggcttgtg agcttgctgt
gtcgtcacaa gagaaagcag cttcctgaag aaaagcagcc
actcctcatg gagaaagagg attaccacag cttgtatcag
agccatttat aaaaggctta ggcaatagag tagggccaaa
aagcctgacc tcactctaac tcaaagtaat gtccaggttc
ccagagaata tctgctggta tttttctgta aagaccattt
gcaaaattgt aacctaatac aaagtgtagc cttcttccaa
ctcaggtaga acacacctgt ctttgtcttg ctgttttcac
tcagcccttt taacattttc ccctaagccc atatgtctaa
ggaaaggatg ctatttggta atgaggaact gttatttgta
tgtgaattaa agtgctctta tttt
SEQ. ID NO. 11
SIZE: 1524 bp
DEFINITION Human melanoma antigen recognized by
T-cells (MART-1) mRNA.
ACCESSION NO.: U06452
agcagacaga ggactctcat taaggaaggt gtcctgtgcc
ctgaccctac aagatgccaa gagaagatgc tcacttcatc
tatggttacc ccaagaaggg gcacggccac tcttacacca
cggctgaaga ggccgctggg atcggcatcc tgacagtgat
cctgggagtc ttactgctca tcggctgttg gtattgtaga
agacgaaatg gatacagagc cttgatggat aaaagtcttc
atgttggcac tcaatgtgcc ttaacaagaa gatgcccaca
agaagggttt gatcatcggg acagcaaagt gtctcttcaa
gagaaaaact gtgaacctgt ggttcccaat gctccacctg
cttatgagaa actctctgca gaacagtcac caccacctta
ttcaccttaa gagccagcga gacacctgag acatgctgaa
attatttctc tcacactttt gcttgaattt aatacagaca
tctaatgttc tcctttggaa tggtgtagga aaaatgcaag
ccatctctaa taataagtca gtgttaaaat tttagtaggt
ccgctagcag tactaatcat gtgaggaaat gatgagaaat
attaaattgg gaaaactcca tcaataaatg ttgcaatgca
tgatactatc tgtgccagag gtaatgttag taaatccatg
gtgttatttt ctgagagaca gaattcaagt gggtattctg
gggccatcca atttctcttt acttgaaatt tggctaataa
caaactagtc aggttttcga accttgaccg acatgaactg
tacacagaat tgttccagta ctatggagtg ctcacaaagg
atacttttac aggttaagac aaagggttga ctggcctatt
tatctgatca agaacatgtc agcaatgtct ctttgtgctc
taaaattcta ttatactaca ataatatatt gtaaagatcc
tatagctctt tttttttgag atggagtttc gcttttgttg
cccaggctgg agtgcaatgg cgcgatcttg gctcaccata
acctccgcct cccaggttca agcaattctc ctgccttagc
ctcctgagta gctgggatta caggcgtgcg ccactatgcc
tgactaattt tgtagtttta gtagagacgg ggtttctcca
tgttggtcag gctggtctca aactcctgac ctcaggtgat
ctgcccgcct cagcctccca aagtgctgga attacaggcg
tgagccacca cgcctggctg gatcctatat cttaggtaag
acatataacg cagtctaatt acatttcact tcaaggctca
atgctattct aactaatgac aagtattttc tactaaacca
gaaattggta gaaggattta aataagtaaa agctactatg
tactgcctta gtgctgatgc ctgtgtactg ccttaaatgt
acctatggca atttagctct cttgggttcc caaatccctc
tcacaagaat gtgcagaaga aatcataaag gatcagagat
tctg
SEQ. ID NO. 12
SIZE: 1466 bp
DEFINITION Homo sapiens synovial sarcoma, X
breakpoint 2 (SSX2), transcript variant 1, mRNA.
ACCESSION NO.: NM_003147
gcatgctctg actttctctc tctttcgatt cttccatact
cagagtacgc acggtctgat tttctctttg gattcttcca
aaatcagagt cagactgctc ccggtgccat gaacggagac
gacgcctttg caaggagacc cacggttggt gctcaaatac
cagagaagat ccaaaaggcc ttcgatgata ttgccaaata
cttctctaag gaagagtggg aaaagatgaa agcctcggag
aaaatcttct atgtgtatat gaagagaaag tatgaggcta
tgactaaact aggtttcaag gccaccctcc cacctttcat
gtgtaataaa cgggccgaag acttccaggg gaatgatttg
gataatgacc ctaaccgtgg gaatcaggtt gaacgtcctc
agatgacttt cggcaggctc cagggaatct ccccgaagat
catgcccaag aagccagcag aggaaggaaa tgattcggag
gaagtgccag aagcatctgg cccacaaaat gatgggaaag
agctgtgccc cccgggaaaa ccaactacct ctgagaagat
tcacgagaga tctggaaata gggaggccca agaaaaggaa
gagagacgcg gaacagctca tcggtggagc agtcagaaca
cacacaacat tggtcgattc agtttgtcaa cttctatggg
tgcagttcat ggtaccccca aaacaattac acacaacagg
gacccaaaag gggggaacat gcctggaccc acagactgcg
tgagagaaaa cagctggtga tttatgaaga gatcagcgac
cctgaggaag atgacgagta actcccctca gggatacgac
acatgcccat gatgagaagc agaacgtggt gacctttcac
gaacatgggc atggctgcgg acccctcgtc atcaggtgca
tagcaagtga aagcaagtgt tcacaacagt gaaaagttga
gcgtcatttt tcttagtgtg ccaagagttc gatgttagcg
tttacgttgt attttcttac actgtgtcat tctgttagat
actaacattt tcattgatga gcaagacata cttaatgcat
attttggttt gtgtatccat gcacctacct tagaaaacaa
gtattgtcgg ttacctctgc atggaacagc attaccctcc
tctctcccca gatgtgacta ctgagggcag ttctgagtgt
ttaatttcag attttttcct ctgcatttac acacacacgc
acacaaacca caccacacac acacacacac acacacacac
acacacacac acacaccaag taccagtata agcatctgcc
atctgctttt cccattgcca tgcgtcctgg tcaagctccc
ctcactctgt ttcctggtca gcatgtactc ccctcatccg
attcccctgt agcagtcact gacagttaat aaacctttgc
aaacgttcaa aaaaaaaaaa aaaaaa
SEQ. ID NO. 13
SIZE: 2653 bp
DEFINITION Homo sapiens folate hydrolase
(prostate-specific membrane antigen) 1 (FOLH1),
mRNA.
ACCESSION NO.: NM_004476
ctcaaaaggg gccggatttc cttctcctgg aggcagatgt
tgcctctctc tctcgctcgg attggttcag tgcactctag
aaacactgct gtggtggaga aactggaccc caggtctgga
gcgaattcca gcctgcaggg ctgataagcg aggcattagt
gagattgaga gagactttac cccgccgtgg tggttggagg
gcgcgcagta gagcagcagc acaggcgcgg gtcccgggag
gccggctctg ctcgcgccga gatgtggaat ctccttcacg
aaaccgactc ggctgtggcc accgcgcgcc gcccgcgctg
gctgtgcgct ggggcgctgg tgctggcggg tggcttcttt
ctcctcggct tcctcttcgg gtggtttata aaatcctcca
atgaagctac taacattact ccaaagcata atatgaaagc
atttttggat gaattgaaag ctgagaacat caagaagttc
ttatataatt ttacacagat accacattta gcaggaacag
aacaaaactt tcagcttgca aagcaaattc aatcccagtg
gaaagaattt ggcctggatt ctgttgagct agcacattat
gatgtcctgt tgtcctaccc aaataagact catcccaact
acatctcaat aattaatgaa gatggaaatg agattttcaa
cacatcatta tttgaaccac ctcctccagg atatgaaaat
gtttcggata ttgtaccacc tttcagtgct ttctctcctc
aaggaatgcc agagggcgat ctagtgtatg ttaactatgc
acgaactgaa gacttcttta aattggaacg ggacatgaaa
atcaattgct ctgggaaaat tgtaattgcc agatatggga
aagttttcag aggaaataag gttaaaaatg cccagctggc
aggggccaaa ggagtcattc tctactccga ccctgctgac
tactttgctc ctggggtgaa gtcctatcca gatggttgga
atcttcctgg aggtggtgtc cagcgtggaa atatcctaaa
tctgaatggt gcaggagacc ctctcacacc aggttaccca
gcaaatgaat atgcttatag gcgtggaatt gcagaggctg
ttggtcttcc aagtattcct gttcatccaa ttggatacta
tgatgcacag aagctcctag aaaaaatggg tggctcagca
ccaccagata gcagctggag aggaagtctc aaagtgccct
acaatgttgg acctggcttt actggaaact tttctacaca
aaaagtcaag atgcacatcc actctaccaa tgaagtgaca
agaatttaca atgtgatagg tactctcaga ggagcagtgg
aaccagacag atatgtcatt ctgggaggtc accgggactc
atgggtgttt ggtggtattg accctcagag tggagcagct
gttgttcatg aaattgtgag gagctttgga acactgaaaa
aggaagggtg gagacctaga agaacaattt tgtttgcaag
ctgggatgca gaagaatttg gtcttcttgg ttctactgag
tgggcagagg agaattcaag actccttcaa gagcgtggcg
tggcttatat taatgctgac tcatctatag aaggaaacta
cactctgaga gttgattgta caccgctgat gtacagcttg
gtacacaacc taacaaaaga gctgaaaagc cctgatgaag
gctttgaagg caaatctctt tatgaaagtt ggactaaaaa
aagtccttcc ccagagttca gtggcatgcc caggataagc
aaattgggat ctggaaatga ttttgaggtg ttcttccaac
gacttggaat tgcttcaggc agagcacggt atactaaaaa
ttgggaaaca aacaaattca gcggctatcc actgtatcac
agtgtctatg aaacatatga gttggtggaa aagttttatg
atccaatgtt taaatatcac ctcactgtgg cccaggttcg
aggagggatg gtgtttgagc tagccaattc catagtgctc
ccttttgatt gtcgagatta tgctgtagtt ttaagaaagt
atgctgacaa aatctacagt atttctatga aacatccaca
ggaaatgaag acatacagtg tatcatttga ttcacttttt
tctgcagtaa agaattttac agaaattgct tccaagttca
gtgagagact ccaggacttt gacaaaagca acccaatagt
attaagaatg atgaatgatc aactcatgtt tctggaaaga
gcatttattg atccattagg gttaccagac aggccttttt
ataggcatgt catctatgct ccaagcagcc acaacaagta
tgcaggggag tcattcccag gaatttatga tgctctgttt
gatattgaaa gcaaagtgga cccttccaag gcctggggag
aagtgaagag acagatttat gttgcagcct tcacagtgca
ggcagctgca gagactttga gtgaagtagc ctaagaggat
tctttagaga atccgtattg aatttgtgtg gtatgtcact
cagaaagaat cgtaatgggt atattgataa attttaaaat
tggtatattt gaaataaagt tgaatattat atataaaaaa
aaaaaaaaaa aaa
SEQ. ID NO. 14
SIZE: 2420 bp
DEFINITION Human antigen (MAGE-1) gene, complete cds.
ACCESSION NO.: M77481
ggatccaggc cctgccagga aaaatataag ggccctgcgt
gagaacagag ggggtcatcc actgcatgag agtggggatg
tcacagagtc cagcccaccc tcctggtagc actgagaagc
cagggctgtg cttgcggtct gcaccctgag ggcccgtgga
ttcctcttcc tggagctcca ggaaccaggc agtgaggcct
tggtctgaga cagtatcctc aggtcacaga gcagaggatg
cacagggtgt gccagcagtg aatgtttgcc ctgaatgcac
accaagggcc ccacctgcca caggacacat aggactccac
agagtctggc ctcacctccc tactgtcagt cctgtagaat
cgacctctgc tggccggctg taccctgagt accctctcac
ttcctccttc aggttttcag gggacaggcc aacccagagg
acaggattcc ctggaggcca cagaggagca ccaaggagaa
gatctgtaag taggcctttg ttagagtctc caaggttcag
ttctcagctg aggcctctca cacactccct ctctccccag
gcctgtgggt cttcattgcc cagctcctgc ccacactcct
gcctgctgcc ctgacgagag tcatcatgtc tcttgagcag
aggagtctgc actgcaagcc tgaggaagcc cttgaggccc
aacaagaggc cctgggcctg gtgtgtgtgc aggctgccac
ctcctcctcc tctcctctgg tcctgggcac cctggaggag
gtgcccactg ctgggtcaac agatcctccc cagagtcctc
agggagcctc cgcctttccc actaccatca acttcactcg
acagaggcaa cccagtgagg gttccagcag ccgtgaagag
gaggggccaa gcacctcttg tatcctggag tccttgttcc
gagcagtaat cactaagaag gtggctgatt tggttggttt
tctgctcctc aaatatcgag ccagggagcc agtcacaaag
gcagaaatgc tggagagtgt catcaaaaat tacaagcact
gttttcctga gatcttcggc aaagcctctg agtccttgca
gctggtcttt ggcattgacg tgaaggaagc agaccccacc
ggccactcct atgtccttgt cacctgccta ggtctctcct
atgatggcct gctgggtgat aatcagatca tgcccaagac
aggcttcctg ataattgtcc tggtcatgat tgcaatggag
ggcggccatg ctcctgagga ggaaatctgg gaggagctga
gtgtgatgga ggtgtatgat gggagggagc acagtgccta
tggggagccc aggaagctgc tcacccaaga tttggtgcag
gaaaagtacc tggagtaccg gcaggtgccg gacagtgatc
ccgcacgcta tgagttcctg tggggtccaa gggccctcgc
tgaaaccagc tatgtgaaag tccttgagta tgtgatcaag
gtcagtgcaa gagttcgctt tttcttccca tccctgcgtg
aagcagcttt gagagaggag gaagagggag tctgagcatg
agttgcagcc aaggccagtg ggagggggac tgggccagtg
caccttccag ggccgcgtcc agcagcttcc cctgcctcgt
gtgacatgag gcccattctt cactctgaag agagcggtca
gtgttctcag tagtaggttt ctgttctatt gggtgacttg
gagatttatc tttgttctct tttggaattg ttcaaatgtt
tttttttaag ggatggttga atgaacttca gcatccaagt
ttatgaatga cagcagtcac acagttctgt gtatatagtt
taagggtaag agtcttgtgt tttattcaga ttgggaaatc
cattctattt tgtgaattgg gataataaca gcagtggaat
aagtacttag aaatgtgaaa aatgagcagt aaaatagatg
agataaagaa ctaaagaaat taagagatag tcaattcttg
ccttatacct cagtctattc tgtaaaattt ttaaagatat
atgcatacct ggatttcctt ggcttctttg agaatgtaag
agaaattaaa tctgaataaa gaattcttcc tgttcactgg
ctcttttctt ctccatgcac tgagcatctg ctttttggaa
ggccctgggt tagtagtgga gatgctaagg taagccagac
tcatacccac ccatagggtc gtagagtcta ggagctgcag
tcacgtaatc gaggtggcaa gatgtcctct aaagatgtag
ggaaaagtga gagaggggtg agggtgtggg gctccgggtg
agagtggtgg agtgtcaatg ccctgagctg gggcattttg
ggctttggga aactgcagtt ccttctgggg gagctgattg
taatgatctt gggtggatcc
SEQ. ID NO. 15
SIZE: 4204 bp DNA linear PRI 07-APR-1994
DEFINITION Human MAGE-3 antigen (MAGE-3) gene,
complete cds.
ACCESSION NO.: U03735
acgcaggcag tgatgtcacc cagaccacac cccttccccc
aatgccactt cagggggtac tcagagtcag agacttggtc
tgaggggagc agaagcaatc tgcagaggat ggcggtccag
gctcagccag gcatcaactt caggaccctg agggatgacc
gaaggccccg cccacccacc cccaactccc ccgaccccac
caggatctac agcctcagga cccccgtccc aatccttacc
ccttgcccca tcaccatctt catgcttacc tccaccccca
tccgatcccc atccaggcag aatccagttc cacccctgcc
cggaacccag ggtagtaccg ttgccaggat gtgacgccac
tgacttgcgc attggaggtc agaagaccgc gagattctcg
ccctgagcaa cgagcgacgg cctgacgtcg gcggagggaa
gccggcccag gctcggtgag gaggcaaggt aagacgctga
gggaggactg aggcgggcct cacctcagac agagggcctc
aaataatcca gtgctgcctc tgctgccggg cctgggccac
cccgcagggg aagacttcca ggctgggtcg ccactacctc
accccgccga cccccgccgc tttagccacg gggaactctg
gggacagagc ttaatgtggc cagggcaggg ctggttagaa
gaggtcaggg cccacgctgt ggcaggaatc aaggtcagga
ccccgagagg gaactgaggg cagcctaacc accaccctca
ccaccattcc cgtcccccaa cacccaaccc cacccccatc
ccccattccc atccccaccc ccacccctat cctggcagaa
tccgggcttt gcccctggta tcaagtcacg gaagctccgg
gaatggcggc caggcacgtg agtcctgagg ttcacatcta
cggctaaggg agggaagggg ttcggtatcg cgagtatggc
cgttgggagg cagcgaaagg gcccaggcct cctggaagac
agtggagtcc tgaggggacc cagcatgcca ggacaggggg
cccactgtac ccctgtctca aaccgaggca ccttttcatt
cggctacggg aatcctaggg atgcagaccc acttcagcag
ggggttgggg cccagccctg cgaggagtca tggggaggaa
gaagagggag gactgagggg accttggagt ccagatcagt
ggcaaccttg ggctggggga tgctgggcac agtggccaaa
tgtgctctgt gctcattgcg ccttcagggt gaccagagag
ttgagggctg tggtctgaag agtgggactt caggtcagca
gagggaggaa tcccaggatc tgcagggccc aaggtgtacc
cccaaggggc ccctatgtgg tggacagatg cagtggtcct
aggatctgcc aagcatccag gtgaagagac tgagggagga
ttgagggtac ccctgggaca gaatgcggac tgggggcccc
ataaaaatct gccctgctcc tgctgttacc tcagagagcc
tgggcagggc tgtcagctga ggtccctcca ttatcctagg
atcactgatg tcagggaagg ggaagccttg gtctgagggg
gctgcactca gggcagtaga gggaggctct cagaccctac
taggagtgga ggtgaggacc aagcagtctc ctcacccagg
gtacatggac ttcaataaat ttggacatct ctcgttgtcc
tttccgggag gacctgggaa tgtatggcca gatgtgggtc
ccctcatgtt tttctgtacc atatcaggta tgtgagttct
tgacatgaga gattctcagg ccagcagaag ggagggatta
ggccctataa ggagaaaggt gagggccctg agtgagcaca
gaggggatcc tccaccccag tagagtgggg acctcacaga
gtctggccaa ccctcctgac agttctggga atccgtggct
gcgtttgctg tctgcacatt gggggcccgt ggattcctct
cccaggaatc aggagctcca ggaacaaggc agtgaggact
tggtctgagg cagtgtcctc aggtcacaga gtagaggggg
ctcagatagt gccaacggtg aaggtttgcc ttggattcaa
accaagggcc ccacctgccc cagaacacat ggactccaga
gcgcctggcc tcaccctcaa tactttcagt cctgcagcct
cagcatgcgc tggccggatg taccctgagg tgccctctca
cttcctcctt caggttctga ggggacaggc tgacctggag
gaccagaggc ccccggagga gcactgaagg agaagatctg
taagtaagcc tttgttagag cctccaaggt tccattcagt
actcagctga ggtctctcac atgctccctc tctccccagg
ccagtgggtc tccattgccc agctcctgcc cacactcccg
cctgttgccc tgaccagagt catcatgcct cttgagcaga
ggagtcagca ctgcaagcct gaagaaggcc ttgaggcccg
aggagaggcc ctgggcctgg tgggtgcgca ggctcctgct
actgaggagc aggaggctgc ctcctcctct tctactctag
ttgaagtcac cctgggggag gtgcctgctg ccgagtcacc
agatcctccc cagagtcctc agggagcctc cagcctcccc
actaccatga actaccctct ctggagccaa tcctatgagg
actccagcaa ccaagaagag gaggggccaa gcaccttccc
tgacctggag tccgagttcc aagcagcact cagtaggaag
gtggccgagt tggttcattt tctgctcctc aagtatcgag
ccagggagcc ggtcacaaag gcagaaatgc tggggagtgt
cgtcggaaat tggcagtatt tctttcctgt gatcttcagc
aaagcttcca gttccttgca gctggtcttt ggcatcgagc
tgatggaagt ggaccccatc ggccacttgt acatctttgc
cacctgcctg ggcctctcct acgatggcct gctgggtgac
aatcagatca tgcccaaggc aggcctcctg ataatcgtcc
tggccataat cgcaagagag ggcgactgtg cccctgagga
gaaaatctgg gaggagctga gtgtgttaga ggtgtttgag
gggagggaag acagtatctt gggggatccc aagaagctgc
tcacccaaca tttcgtgcag gaaaactacc tggagtaccg
gcaggtcccc ggcagtgatc ctgcatgtta tgaattcctg
tggggtccaa gggccctcgt tgaaaccagc tatgtgaaag
tcctgcacca tatggtaaag atcagtggag gacctcacat
ttcctaccca cccctgcatg agtgggtttt gagagagggg
gaagagtgag tctgagcacg agttgcagcc agggccagtg
ggagggggtc tgggccagtg caccttccgg ggccgcatcc
cttagtttcc actgcctcct gtgacgtgag gcccattctt
cactctttga agcgagcagt cagcattctt agtagtgggt
ttctgttctg ttggatgact ttgagattat tctttgtttc
ctgttggagt tgttcaaatg ttccttttaa cggatggttg
aatgagcgtc agcatccagg tttatgaatg acagtagtca
cacatagtgc tgtttatata gtttaggagt aagagtcttg
ttttttactc aaattgggaa atccattcca ttttgtgaat
tgtgacataa taatagcagt ggtaaaagta tttgcttaaa
attgtgagcg aattagcaat aacatacatg agataactca
agaaatcaaa agatagttga ttcttgcctt gtacctcaat
ctattctgta aaattaaaca aatatgcaaa ccaggatttc
cttgacttct ttgagaatgc aagcgaaatt aaatctgaat
aaataattct tcctcttcac tggctcgttt cttttccgtt
cactcagcat ctgctctgtg ggaggccctg ggttagtagt
ggggatgcta aggtaagcca gactcacgcc tacccatagg
gctgtagagc ctaggacctg cagtcatata attaaggtgg
tgagaagtcc tgtaagatgt agaggaaatg taagagaggg
gtgagggtgt ggcgctccgg gtgagagtag tggagtgtca
gtgc
SEQ. ID NO. 16
SIZE: 752 bp
DEFINITION Human autoimmunogenic cancer/testis
antigen NY-ESO-1 mRNA, complete cds.
ACCESSION NO.: U87459
atcctcgtgg gccctgacct tctctctgag agccgggcag
aggctccgga gccatgcagg ccgaaggccg gggcacaggg
ggttcgacgg gcgatgctga tggcccagga ggccctggca
ttcctgatgg cccagggggc aatgctggcg gcccaggaga
ggcgggtgcc acgggcggca gaggtccccg gggcgcaggg
gcagcaaggg cctcggggcc gggaggaggc gccccgcggg
gtccgcatgg cggcgcggct tcagggctga atggatgctg
cagatgcggg gccagggggc cggagagccg cctgcttgag
ttctacctcg ccatgccttt cgcgacaccc atggaagcag
agctggcccg caggagcctg gcccaggatg ccccaccgct
tcccgtgcca ggggtgcttc tgaaggagtt cactgtgtcc
ggcaacatac tgactatccg actgactgct gcagaccacc
gccaactgca gctctccatc agctcctgtc tccagcagct
ttccctgttg atgtggatca cgcagtgctt tctgcccgtg
tttttggctc agcctccctc agggcagagg cgctaagccc
agcctggcgc cccttcctag gtcatgcctc ctcccctagg
gaatggtccc agcacgagtg gccagttcat tgtgggggcc
tgattgtttg tcgctggagg aggacggctt acatgtttgt
ttctgtagaa aataaaactg agctacgaaa aa
SEQ. ID NO. 17
SIZE: 2148 bp
DEFINITION Homo sapiens preferentially expressed
antigen in melanoma (PRAME), mRNA.
ACCESSION NO.: NM_006115
gcttcagggt acagctcccc cgcagccaga agccgggcct
gcagcgcctc agcaccgctc cgggacaccc cacccgcttc
ccaggcgtga cctgtcaaca gcaacttcgc ggtgtggtga
actctctgag gaaaaaccat tttgattatt actctcagac
gtgcgtggca acaagtgact gagacctaga aatccaagcg
ttggaggtcc tgaggccagc ctaagtcgct tcaaaatgga
acgaaggcgt ttgtggggtt ccattcagag ccgatacatc
agcatgagtg tgtggacaag cccacggaga cttgtggagc
tggcagggca gagcctgctg aaggatgagg ccctggccat
tgccgccctg gagttgctgc ccagggagct cttcccgcca
ctcttcatgg cagcctttga cgggagacac agccagaccc
tgaaggcaat ggtgcaggcc tggcccttca cctgcctccc
tctgggagtg ctgatgaagg gacaacatct tcacctggag
accttcaaag ctgtgcttga tggacttgat gtgctccttg
cccaggaggt tcgccccagg aggtggaaac ttcaagtgct
ggatttacgg aagaactctc atcaggactt ctggactgta
tggtctggaa acagggccag tctgtactca tttccagagc
cagaagcagc tcagcccatg acaaagaagc gaaaagtaga
tggtttgagc acagaggcag agcagccctt cattccagta
gaggtgctcg tagacctgtt cctcaaggaa ggtgcctgtg
atgaattgtt ctcctacctc attgagaaag tgaagcgaaa
gaaaaatgta ctacgcctgt gctgtaagaa gctgaagatt
tttgcaatgc ccatgcagga tatcaagatg atcctgaaaa
tggtgcagct ggactctatt gaagatttgg aagtgacttg
tacctggaag ctacccacct tggcgaaatt ttctccttac
ctgggccaga tgattaatct gcgtagactc ctcctctccc
acatccatgc atcttcctac atttccccgg agaaggaaga
gcagtatatc gcccagttca cctctcagtt cctcagtctg
cagtgcctgc aggctctcta tgtggactct ttatttttcc
ttagaggccg cctggatcag ttgctcaggc acgtgatgaa
ccccttggaa accctctcaa taactaactg ccggctttcg
gaaggggatg tgatgcatct gtcccagagt cccagcgtca
gtcagctaag tgtcctgagt ctaagtgggg tcatgctgac
cgatgtaagt cccgagcccc tccaagctct gctggagaga
gcctctgcca ccctccagga cctggtcttt gatgagtgtg
ggatcacgga tgatcagctc cttgccctcc tgccttccct
gagccactgc tcccagctta caaccttaag cttctacggg
aattccatct ccatatctgc cttgcagagt ctcctgcagc
acctcatcgg gctgagcaat ctgacccacg tgctgtatcc
tgtccccctg gagagttatg aggacatcca tggtaccctc
cacctggaga ggcttgccta tctgcatgcc aggctcaggg
agttgctgtg tgagttgggg cggcccagca tggtctggct
tagtgccaac ccctgtcctc actgtgggga cagaaccttc
tatgacccgg agcccatcct gtgcccctgt ttcatgccta
actagctggg tgcacatatc aaatgcttca ttctgcatac
ttggacacta aagccaggat gtgcatgcat cttgaagcaa
caaagcagcc acagtttcag acaaatgttc agtgtgagtg
aggaaaacat gttcagtgag gaaaaaacat tcagacaaat
gttcagtgag gaaaaaaagg ggaagttggg gataggcaga
tgttgacttg aggagttaat gtgatctttg gggagataca
tcttatagag ttagaaatag aatctgaatt tctaaaggga
gattctggct tgggaagtac atgtaggagt taatccctgt
gtagactgtt gtaaagaaac tgttgaaaat aaagagaagc
aatgtgaagc aaaaaaaaaa aaaaaaaa
SEQ. ID NO. 18
SIZE: 4530 bp
DEFINITION Human tyrosine kinase-type receptor
(HER2) mRNA, complete cds.
ACCESSION NO.: M11730
aattctcgag ctcgtcgacc ggtcgacgag ctcgagggtc
gacgagctcg agggcgcgcg cccggccccc acccctcgca
gcaccccgcg ccccgcgccc tcccagccgg gtccagccgg
agccatgggg ccggagccgc agtgagcacc atggagctgg
cggccttgtg ccgctggggg ctcctcctcg ccctcttgcc
ccccggagcc gcgagcaccc aagtgtgcac cggcacagac
atgaagctgc ggctccctgc cagtcccgag acccacctgg
acatgctccg ccacctctac cagggctgcc aggtggtgca
gggaaacctg gaactcacct acctgcccac caatgccagc
ctgtccttcc tgcaggatat ccaggaggtg cagggctacg
tgctcatcgc tcacaaccaa gtgaggcagg tcccactgca
gaggctgcgg attgtgcgag gcacccagct ctttgaggac
aactatgccc tggccgtgct agacaatgga gacccgctga
acaataccac ccctgtcaca ggggcctccc caggaggcct
gcgggagctg cagcttcgaa gcctcacaga gatcttgaaa
ggaggggtct tgatccagcg gaacccccag ctctgctacc
aggacacgat tttgtggaag gacatcttcc acaagaacaa
ccagctggct ctcacactga tagacaccaa ccgctctcgg
gcctgccacc cctgttctcc gatgtgtaag ggctcccgct
gctggggaga gagttctgag gattgtcaga gcctgacgcg
cactgtctgt gccggtggct gtgcccgctg caaggggcca
ctgcccactg actgctgcca tgagcagtgt gctgccggct
gcacgggccc caagcactct gactgcctgg cctgcctcca
cttcaaccac agtggcatct gtgagctgca ctgcccagcc
ctggtcacct acaacacaga cacgtttgag tccatgccca
atcccgaggg ccggtataca ttcggcgcca gctgtgtgac
tgcctgtccc tacaactacc tttctacgga cgtgggatcc
tgcaccctcg tctgccccct gcacaaccaa gaggtgacag
cagaggatgg aacacagcgg tgtgagaagt gcagcaagcc
ctgtgcccga gtgtgctatg gtctgggcat ggagcacttg
cgagaggtga gggcagttac cagtgccaat atccaggagt
ttgctggctg caagaagatc tttgggagcc tggcatttct
gccggagagc tttgatgggg acccagcctc caacactgcc
ccgctccagc cagagcagct ccaagtgttt gagactctgg
aagagatcac aggttaccta tacatctcag catggccgga
cagcctgcct gacctcagcg tcttccagaa cctgcaagta
atccggggac gaattctgca caatggcgcc tactcgctga
ccctgcaagg gctgggcatc agctggctgg ggctgcgctc
actgagggaa ctgggcagtg gactggccct catccaccat
aacacccacc tctgcttcgt gcacacggtg ccctgggacc
agctctttcg gaacccgcac caagctctgc tccacactgc
caaccggcca gaggacgagt gtgtgggcga gggcctggcc
tgccaccagc tgtgcgcccg agggcactgc tggggtccag
ggcccaccca gtgtgtcaac tgcagccagt tccttcgggg
ccaggagtgc gtggaggaat gccgagtact gcaggggctc
cccagggagt atgtgaatgc caggcactgt ttgccgtgcc
accctgagtg tcagccccag aatggctcag tgacctgttt
tggaccggag gctgaccagt gtgtggcctg tgcccactat
aaggaccctc ccttctgcgt ggcccgctgc cccagcggtg
tgaaacctga cctctcctac atgcccatct ggaagtttcc
agatgaggag ggcgcatgcc agccttgccc catcaactgc
acccactcct gtgtggacct ggatgacaag ggctgccccg
ccgagcagag agccagccct ctgacgtcca tcgtctctgc
ggtggttggc attctgctgg tcgtggtctt gggggtggtc
tttgggatcc tcatcaagcg acggcagcag aagatccgga
agtacacgat gcggagactg ctgcaggaaa cggagctggt
ggagccgctg acacctagcg gagcgatgcc caaccaggcg
cagatgcgga tcctgaaaga gacggagctg aggaaggtga
aggtgcttgg atctggcgct tttggcacag tctacaaggg
catctggatc cctgatgggg agaatgtgaa aattccagtg
gccatcaaag tgttgaggga aaacacatcc cccaaagcca
acaaagaaat cttagacgaa gcatacgtga tggctggtgt
gggctcccca tatgtctccc gccttctggg catctgcctg
acatccacgg tgcagctggt gacacagctt atgccctatg
gctgcctctt agaccatgtc cgggaaaacc gcggacgcct
gggctcccag gacctgctga actggtgtat gcagattgcc
aaggggatga gctacctgga ggatgtgcgg ctcgtacaca
gggacttggc cgctcggaac gtgctggtca agagtcccaa
ccatgtcaaa attacagact tcgggctggc tcggctgctg
gacattgacg agacagagta ccatgcagat gggggcaagg
tgcccatcaa gtggatggcg ctggagtcca ttctccgccg
gcggttcacc caccagagtg atgtgtggag ttatggtgtg
actgtgtggg agctgatgac ttttggggcc aaaccttacg
atgggatccc agcccgggag atccctgacc tgctggaaaa
gggggagcgg ctgccccagc cccccatctg caccattgat
gtctacatga tcatggtcaa atgttggatg attgactctg
aatgtcggcc aagattccgg gagttggtgt ctgaattctc
ccgcatggcc agggaccccc agcgctttgt ggtcatccag
aatgaggact tgggcccagc cagtcccttg gacagcacct
tctaccgctc actgctggag gacgatgaca tgggggacct
ggtggatgct gaggagtatc tggtacccca gcagggcttc
ttctgtccag accctgcccc gggcgctggg ggcatggtcc
accacaggca ccgcagctca tctaccagga gtggcggtgg
ggacctgaca ctagggctgg agccctctga agaggaggcc
cccaggtctc cactggcacc ctccgaaggg gctggctccg
atgtatttga tggtgacctg ggaatggggg cagccaaggg
gctgcaaagc ctccccacac atgaccccag ccctctacag
cggtacagtg aggaccccac agtacccctg ccctctgaga
ctgatggcta cgttgccccc ctgacctgca gcccccagcc
tgaatatgtg aaccagccag atgttcggcc ccagccccct
tcgccccgag agggccctct gcctgctgcc cgacctgctg
gtgccactct ggaaagggcc aagactctct ccccagggaa
gaatggggtc gtcaaagacg tttttgcctt tgggggtgcc
gtggagaacc ccgagtactt gacaccccag ggaggagctg
cccctcagcc ccaccctcct cctgccttca gcccagcctt
cgacaacctc tattactggg accaggaccc accagagcgg
ggggctccac ccagcacctt caaagggaca cctacggcag
agaacccaga gtacctgggt ctggacgtgc cagtgtgaac
cagaaggcca agtccgcaga agccctgatg tgtcctcagg
gagcagggaa ggcctgactt ctgctggcat caagaggtgg
gagggccctc cgaccacttc caggggaacc tgccatgcca
ggaacctgtc ctaaggaacc ttccttcctg cttgagttcc
cagatggctg gaaggggtcc agcctcgttg gaagaggaac
agcactgggg agtctttgtg gattctgagg ccctgcccaa
tgagactcta gggtccagtg gatgccacag cccagcttgg
ccctttcctt ccagatcctg ggtactgaaa gccttaggga
agctggcctg agaggggaag cggccctaag ggagtgtcta
agaacaaaag cgacccattc agagactgtc cctgaaacct
agtactgccc cccatgagga aggaacagca atggtgtcag
tatccaggct ttgtacagag tgcttttctg tttagttttt
actttttttg ttttgttttt ttaaagacga aataaagacc
caggggagaa tgggtgttgt atggggaggc aagtgtgggg
ggtccttctc cacacccact ttgtccattt gcaaatatat
tttggaaaac

1-53. (canceled)

54. An immunogenic medicament for the treatment of prostate, melanoma, breast, esophageal or renal cancer comprising at least a first antigen and a second antigen associated with prostate, melanoma, breast, esophageal or renal cancers, wherein the first and second antigens are provided separately or in combination, wherein each antigen is 1) a whole antigen, 2) a fragment of an antigen, 3) an epitope cluster derived from an antigen, 4) an epitope derived from an antigen, or 5) a nucleic acid encoding any of 1 to 4; wherein the first antigen is PRAME and the second antigen comprises a tumor-associated antigen, a cancer testis (CT) antigen, or an antigen associated with tumor neovasculature.

55. The medicament of claim 54, wherein the antigen associated with tumor neovasculature is selected from the group consisting of VEGFR2, Tie-2, and PSMA.

56. The medicament of claim 54, wherein the cancer testis antigen is selected from the group consisting of SSX-2, NY-ESO-1, and PSMA.

57. The medicament of claim 54, wherein the cancer testis antigen antigen comprises NY-ESO-1 and SSX-2.

58. The medicament of claim 54, wherein the tumor-associated antigen comprises NY-ESO-1, SSX-2, and PSMA.

59. The medicament of claim 54, wherein the medicament exists as a single formulation comprising said at least first and second antigens.

60. The medicament of claim 54, wherein the medicament comprises at least two separate formulations each comprising one of said first and second antigens.

61. The medicament of claim 54, wherein the first and second antigens are associated with prostate, melanoma, breast, esophageal and renal cancers.

62. An immunogenic medicament for the treatment of prostate, melanoma, breast, esophageal or renal cancer comprising at least a first antigen and a second antigen associated with prostate, melanoma, breast, esophageal or renal cancers, wherein the first and second antigens are provided separately or in combination, wherein each antigen is 1) a whole antigen, 2) a fragment of an antigen, 3) an epitope cluster derived from an antigen, 4) an epitope derived from an antigen, or 5) a nucleic acid encoding any of 1 to 4; wherein the medicament comprises PRAME and at least one other antigen; and wherein the medicament has been adapted for intranodal administration.

63. The medicament of claim 62, wherein the at least one other antigen comprises a tumor-associated antigen.

64. The medicament of claim 62, wherein the at least one other antigen is selected from the group consisting of VEGFR2, Tie-2, SSX-2, NY-ESO-1, and PSMA.

65. The medicament of claim 62, wherein the at least one other antigen comprises a cancer/testis (CT) antigen.

66. The medicament of claim 62, wherein the first and second antigens are associated with prostate, melanoma, breast, esophageal and renal cancers.

67. An immunogenic medicament for the treatment of prostate, melanoma, breast, esophageal or renal cancer comprising an agent that elicits a T cell response to PRAME and to at least one other antigen comprising a tumor-associated antigen or an antigen associated with tumor neovasculature, wherein each antigen is 1) a whole antigen, 2) a fragment of an antigen, 3) an epitope cluster derived from an antigen, 4) an epitope derived from an antigen, or 5) a nucleic acid encoding any of 1 to 4.

68. An immunogenic medicament for the treatment of prostate, melanoma, breast, esophageal or renal cancer comprising a means for inducing a T cell response to PRAME and to at least one other antigen associated with prostate, melanoma, breast, esophageal or renal cancers, the medicament comprising a tumor-associated antigen or an antigen associated with tumor neovasculature; and a physiologically acceptable buffer.

69. An immunogenic medicament for the treatment of prostate, melanoma, breast, esophageal or renal cancer wherein said medicament induces a T cell response to PRAME and to at least one other antigen associated with prostate, melanoma, breast, esophageal or renal cancers, the medicament comprising a tumor-associated antigen or an antigen associated with tumor neovasculature.

70. An immunogenic medicament for the treatment of prostate, melanoma, breast, esophageal or renal cancer comprising at least a first antigen and a second antigen associated with prostate, melanoma, breast, esophageal or renal cancers, wherein the first and second antigens are provided separately or in combination, wherein each antigen is 1) a whole antigen, 2) a fragment of an antigen, 3) an epitope cluster derived from an antigen, 4) an epitope derived from an antigen or 5) a nucleic acid encoding any of 1 to 4; wherein the medicament comprises PRAME and at least one other antigen; and wherein the medicament has been adapted for intralymphatic administration.

71. The medicament of claim 70, wherein the first and second antigens are associated with prostate, melanoma, breast, esophageal and renal cancers.

72. An immunogenic medicament for the treatment of prostate, melanoma, breast, esophageal or renal cancer comprising a means for inducing a T cell response to PRAME and to at least one other antigen associated with prostate, melanoma, breast, esophageal and/or renal cancers, wherein the T cell response comprise a response to a tumor neovasculature antigen; and a physiologically acceptable buffer.
